(19) United States
(12) Patent Application Publication
(10) Pub. No.: US 2009/0105221 A1
(43) Pub. Date: Apr. 23, 2009

(54) MUSCARINIC RECEPTOR ANTAGONISTS

(75) Inventors: Mohammad Salman, Princeton, NJ (US); Pakala Kumara Savithru Sarma, Haryana (IN); Arani Pal, Calcutta (IN); Anita Chugh, Delhi (IN); Suman Gupta, Haryana (IN); Venkata P. Palle, Maharashtra (IN); Naresh Kumar, Gurgaon (IN); Kirandeep Kaur, Haryana (IN)

Correspondence Address: RANBAXY INC., 600 COLLEGE ROAD EAST, SUITE 2100, PRINCETON, NJ, 08540, US

(73) Assignee: RANBAXY LABORATORIES LIMITED, Gurgaon (IN)

(21) Appl. No.: 11/576,096

(22) PCT Filed: Sep. 28, 2005
(86) PCT No.: PCT/IB2005/002901

§ 371 Date: Oct. 1, 2008

Related U.S. Application Data
(60) Provisional application No. 60/614,186, filed on Sep. 29, 2004.

(51) Int. Cl. A61K 31/55; C07D 223/14; C07D 405/12; A61P 11/00; A61P 1/00

(52) U.S. Cl. 514/216; 540/585

(57) ABSTRACT

This present invention generally relates to muscarinic receptor antagonists of Formula (I), which are useful, among other uses, for the treatment of various diseases of the respiratory, urinary and gastrointestinal systems mediated through muscarinic receptors. The invention also relates to the process for the prepration of disclosed compounds, pharmaceutical compositions containing the disclosed compounds, and the methods for treating diseases mediated through muscarinic receptors.

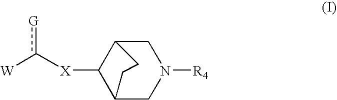

### FIELD OF THE INVENTION

This present invention generally relates to muscarinic receptor antagonists which are useful, among other uses, for the treatment of various diseases of the respiratory, urinary and gastrointestinal systems mediated through muscarinic receptors. The invention also relates to the process for the prepration of disclosed compounds, pharmaceutical compositions containing the disclosed compounds, and the methods for treating diseases mediated through muscarinic receptors.

### BACKGROUND OF THE INVENTION

Physiological effects elicited by the neurotransmitter acetylcholine are mediated through its interaction with two major classes of acetylcholine receptors—the nicotinic and muscarinic acetylcholine receptors. Muscarinic receptors belong to the superfamily of G-protein coupled receptors and five molecularly distinct subtypes are known to exist (M₁, M₂, M₃, M₄ and M₅).

These receptors are widely distributed on multiple organs and tissues and are critical to the maintenance of central and peripheral cholinergic neurotransmission. The regional distribution of these receptor sub-types in the brain and other organs has been documented. (for example, the M₁ subtype is located primarily in neuronal tissues such as cereberal cortex and autonomic ganglia, the M₂ subtype is present mainly in the heart and bladder smooth muscle, and the M₃ subtype is located predominantly on smooth muscle and salivary glands (Nature, 323, p. 411 (1986); Science, 237, p. 527 (1987)).

A review in Curr. Opin. Chem. Biol., 3, p. 426 (1999), as well as in Trends in Pharmacol. Sci., 22, p. 409 (2001) by Eglen et. al., describes the biological potentials of modulating muscarinic receptor subtypes by ligands in different disease conditions, such as Alzheimer's disease, pain, urinary disease condition, chronic obstructive pulmonary disease, and the like. The pharmacological and medical aspects of the muscarinic class of acetylcholine agonists and antagonists are presented in a review in Molecules, 6, p. 142 (2001).

Birdsall et. al. in Trends in Pharmacol. Sci., 22, p. 215 (2001) has also summarized the recent developments on the role of different muscarinic receptor subtypes using different muscarinic receptor of knock out mice.

Almost all the smooth muscles express a mixed population of M₂ and M₃ receptors. Although the M₂-receptors are the predominant cholinoreceptors, the smaller population of M₃-receptors appears to be the most functionally important as they mediate the direct contraction of these smooth muscles. Muscarinic receptor antagonists are known to be useful for treating various medical conditions associated with improper smooth muscle function, such as overactive bladder syndrome, irritable bowel syndrome and chronic obstructive pulmonary disease. However the therapeutic utility of antimuscarinics has been limited by poor tolerability as a result of treatment related, frequent systemic adverse events such as dry mouth, constipation, blurred vision, headache, somnolence and tachycardia. Thus, there exists a need for novel muscarinic receptor antagonists that demonstrate target organ selectivity.

Compounds having antagonistic activity against muscarinic receptors have been described in Japanese patent application Laid Open Number 92921/1994 and 135958/1994; WO 93/16048; U.S. Pat. No. 3,176,019; GB 940,540; EP 0325 571; WO 98/29402; EP 0801067; EP 0388054; WO 9109013; U.S. Pat. No. 5,281,601. Also, U.S. Pat. Nos. 6,174,900, 6,130,232 and 5,948,792; WO 97/45414 are related to 1,4-disubstituted piperidine derivatives; WO 98/05641 describes fluorinated, 1,4-disubstitued piperidine derivatives; WO 93/16018 and W096/33973 are other references of interest. U.S. Pat. No. 5,397,800 discloses 1-azabicyclo[2.2.1]heptanes. U.S. Pat. No. 5,001,160 describes 1-aryl-1-hydroxy-1-substituted-3-(4-substituted-1-piperazinyl)-2-propanones. WO 01/42213 describes 2-biphenyl-4-piperidinyl ureas. WO 01/42212 describes carbamate derivatives. WO 01/90081 describes amino alkyl lactam. WO 02/53564 describes quinuclidine derivatives. WO 02/00652 describes carbamates derived from arylalkyl amines. WO 02/06241 describes 1,2,3,5-tetrahydrobenzo(c)azepin-4-one derivatives. U.S. application No. 20030105071 describes thiazole and other heterocyclic ligands as useful for mammalian dopamine, muscarinic and serotonic receptors and transporters, and method of use thereof. WO 04/005252 discloses azabicyclo derivatives described as musacrinic receptor antagonists. WO 04/004629 discloses 3,6-disubstituted azabicyclo [3.1.0] hexane derivatives described as useful muscarinic receptor antagonists. WO 01/47893 describes azabicycloctane derivatives said to be useful in the treatment of cardiac arrhythmias. WO 99/43657 describes 2-arylethyl-(piperidin-4-ylmethyl)amine derivatives reportedly as muscarinic receptor antagonists. WO 01/090082 describes substituted 1-amino-alkyl lactams and their apparent use as muscarinic receptor antagonists. WO 03/033495 describes quinuclidine derivatives and their putative use as M₂ and/or M₃ muscarinic receptor antagonists. U.S. application 2003 0171362 describes amino-tetralin derivatives said to be muscarinic receptor antagonists. U.S. application 20030162780 describes 4-piperidinyl alkyl amine derivatives as muscarinic receptor antagonists. WO 04/014853 and WO 04/014363 disclose derivatives of 3,6-disubstituted azabicyclohexane said to be useful as muscarinic receptor antagonists.

WO 04/052857 and WO 04/067510 disclose 3,6-disubstituted azabicyclo [3.1.0] hexane derivatives described as useful muscarinic receptor antagonists. WO 04/056811 discloses flaxavate derivatives as muscarinic receptor antagonists. WO 04/056810 discloses xanthene derivatives as muscarinic receptor antagonists. WO 04/056767 discloses 1-substituted-3-pyrrolidine derivatives as muscarinic receptor antagonists. WO 04/089363, WO 04/089898, WO 04/069835, WO 04/089900 and WO 04/089364 disclose substituted azabicyclohexane derivatives as muscarinic receptor antagonists.

J. Med. Chem., 44, p. 984 (2002), describes cyclohexylmethylpiperidinyl-triphenylpropioamide derivatives as selective M₃ antagonist discriminating against the other receptor subtypes. J. Med. Chem., 36, p. 610 (1993), describes the synthesis and antimuscarinic activity of some 1-cycloalkyl-1-hydroxy-1-phenyl-3-(4-substituted piperazinyl)-2-propanones and related compounds. J. Med. Chem., 34, p.3065 (1991), describes analogues of oxybutynin, synthesis and antimuscarinic activity of some substituted 7-amino-1-hydroxy-5-heptyn-2-ones and related compounds. Bio-Org. Med. Chem. Lett., 15, 2093 (2005) describes the synthesis and activity of analogues of oxybutynin and tolterodine. The present invention fills the need of muscarinic receptor antagonists useful in the treatment of disease states associated with improper smooth muscle function and respiratory disorders.

### SUMMARY OF THE INVENTION

In one aspect, there are provided muscarinic receptor antagonists, which can be useful as safe and effective therapeutic or prophylactic agents for the treatment of various diseases of the respiratory, urinary and gastrointestinal systems. Also provided are processes for synthesizing such compounds.

In another aspect, pharmaceutical compositions containing such compounds are provided together with acceptable carriers, excipients or diluents which can be useful for the treatment of various diseases of the respiratory, urinary and gastrointestinal systems.

The enantiomers, diastereomers, N-oxides, polymorphs, pharmaceutically acceptable salts and pharmaceutically acceptable solvates of these compounds as well as metabolites having the same type of activity are also provided, as well as pharmaceutical compositions comprising the compounds, their metabolites, enantiomers, diastereomers, N-oxides, polymorphs, solvates or pharmaceutically acceptable salts thereof, in combination with a pharmaceutically acceptable carrier and optionally included excipients.

Other aspects will be set forth in the description which follows, and in part will be apparent from the description or may be learnt by the practice of the invention.

In accordance with one aspect, there are provided compounds having the structure of Formula I:

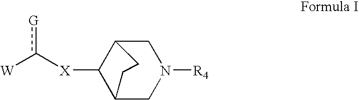

[symbol]

represents an optional double bond;
W can represent

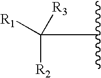

heteroaryl, or heterocyclyl, where [symbol] represents a point of attachment.
R₁ can be aryl, heteroaryl, heterocyclyl, or cycloalkyl.
R₂ can be alkyl, alkenyl, alkynyl, cycloalkyl, aryl, heteroaryl, heterocyclyl, heteroarylalkyl, or heterocyclylalkyl.
R₃ can be hydrogen, lower alkyl, hydroxy, alkoxy, alkenyloxy, alkynyloxy, halogen, or amino.
X can be oxygen, sulphur, or alkylene wherein the alkylene group may be interrupted by 1-5 oxygen, sulfur and —NRₐ (where Rₐ can be hydrogen, alkyl, cycloalkyl, alkenyl, alkynyl, or aryl) groups.
R₄ can be hydrogen; alkyl; alkenyl; alkynyl; cycloalkyl; carboxy; halogen; aryl; aralkyl; acyl; heteroaryl; heterocyclyl; SO₂R₅ [wherein R₅ is selected from alkyl, alkenyl, alkynyl, cycloalkyl, —NRₚR_{q} (wherein Rₚ and R_{q} are selected from hydrogen, alkyl, alkenyl, alkynyl, cycloalkyl, aryl, aralkyl, heterocyclyl, heteroaryl, heterocyclylalkyl, or heteroarylalkyl, or Rₚ and R_{q} may also together join to form a heterocyclyl ring), aryl, aralkyl, heteroaryl, heterocyclyl, heterocyclylalkyl, or heteroarylalkyl]; —COOR₆ (wherein R₆ is selected from alkyl, alkenyl, alkynyl, cycloalkyl, aryl, or aralkyl); —(C═O)NRₓR_{y} [wherein Rₓ and R_{y} are selected from hydrogen, hydroxy (as restricted by the definition that both Rₓ and R_{y} cannot be hydroxy at the same time), alkyl, alkenyl, alkynyl, aryl, aralkyl, —SO₂R₅ (wherein R₅ is the same as defined above), heteroaryl, heterocyclyl, heteroarylalkyl, or heterocyclylalkyl, or Rₓ and R_{y} may also together join to form a heterocyclyl ring]; —NRₓR_{y} wherein Rₓ and R_{y} are the same as defined above; and —OC(═O)NRₓR_{y} (wherein Rₓ and R_{y} are the same as defined above).

G can be —OR (wherein R represents hydrogen or unsubstituted lower (C₁-C₆) alkyl); —NOR (wherein R is the same as defined above); —NHYR′ (wherein R′ is hydrogen, alkyl or aryl and Y is —C(═O), SO, SO₂); or oxygen. The compounds of Formula I are also restricted by the definition that when X is oxygen or sulphur, then G can not be OR (wherein R is the same as defined above).

The following definitions apply to terms as used herein.

The term “alkyl,” unless otherwise specified, refers to a monoradical branched or unbranched saturated hydrocarbon chain having from 1 to 20 carbon atoms. This term is exemplified by groups such as methyl, ethyl, n-propyl, iso-propyl, n-butyl, iso-butyl, t-butyl, n-hexyl, n-decyl, tetradecyl, and the like. Alkyl may further be substituted with one or more substituents selected from alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, —COOR₆ (wherein R₆ is as defined earlier), arylthio, thiol, alkylthio, aryloxy, aminosulfonyl, aminocarbonylamino, —NRₓR_{y}, —C(═O)NRₓR_{y}, —OC(═O)NRₓR_{y} (wherein Rₓ and R_{y} are as defined earlier), nitro, —S(O)ₙR₅ (wherein R₅ is the same as defined earlier and n is 0, 1 or 2). Unless otherwise constrained by the definition, all substituents may be further substituted by 1-3 substituents chosen from alkyl, carboxy, —COOR₆ (wherein R₆ is as defined earlier), —NRₓR_{y}, —C(═O)NRₓR_{y}, —OC(═O)NRₓR_{y} (wherein Rₓ and R_{y} are as defined earlier), hydroxy, alkoxy, halogen, —CF₃, cyano, and —S(O)ₙR₅ (where n and R₅ are as defined earlier).

Alkyl groups may also be interrupted by 1-5 atoms of groups independently chosen from oxygen, sulfur and —NRₐ (where Rₐ is chosen from hydrogen, alkyl, cycloalkyl, alkenyl, alkynyl, aryl). Unless otherwise constrained by the definition, all substituents may be further substituted by 1-3 substituents chosen from alkyl, carboxy, —COOR₆ (wherein R₆ is as defined earlier), —NRₓR_{y}, —C(═O)NRₓR_{y}, —OC(═O)NRₓR_{y} (wherein Rₓ and R_{y} are as defined earlier), hydroxy, alkoxy, halogen, CF₃, cyano, and —S(O)ₙR₅ (where n and R₅ are as defined earlier).

The term “alkylene,” unless otherwise specified, refers to a diradical branched or unbranched saturated hydrocarbon chain having from 1 to 6 carbon atoms. This term can be exemplified by groups such as methylene, ethylene, propylene isomers and the like.

Alkylene may further be substituted with one or more substituents selected from alkyl, alkenyl, alkynyl, alkoxy, cycloalkyl, acyl, acylamino, acyloxy, alkoxycarbonylamino, azido, cyano, halogen, hydroxy, oxo, thiocarbonyl, carboxy, arylthio, thiol, alkylthio, aryloxy, heteroaryloxy, aminosulfonyl, —COOR₆ (wherein R₆ is as defined earlier), —NHC(═O)Rₓ, —NRₓR_{y}, —C(═O)NRₓR_{y}, —NHC(═O)NRₓR_{y}, —C(═O)heteroaryl, —C(═O)heterocyclyl, —OC(═O)NRₓR_{y} (wherein Rₓ and R_{y} are as defined earlier), nitro, —S(O)ₙR₅ (wherein n and R₅ are as defined earlier). Unless otherwise constrained by the definition, all substituents may be further substituted by 1-3 substituents chosen from alkyl, carboxy, —COOR₆ (wherein R₆ is as defined earlier), —NRₓR_{y}, —C(═O)NRₓR_{y}, —OC(═O)NRₓR_{y}, —NHC(═O)NRₓR_{y} (wherein Rₓ and R_{y} are as defined earlier), hydroxy, alkoxy, halogen, CF₃, cyano, and —S(O)ₘR₅ (where R₅ and n are as defined earlier).

Alkylene groups may also be interrupted by 1-5 atoms chosen from oxygen, sulfur and —NRₐ (where Rₐ is chosen from hydrogen, alkyl, cycloalkyl, alkenyl, alkynyl, aryl). Unless otherwise constrained by the definition, all substituents may be further substituted by 1-3 substituents chosen from alkyl, carboxy, —COOR₆ (wherein R₆ is as defined earlier), —NRₓR_{y}, —C(═O)NRₓR_{y}, —O—C(═O)NRₓR_{y} (wherein Rₓ and R_{y} are as defined earlier), hydroxy, alkoxy, halogen, CF₃, cyano, and —S(O)ₙR₅ (where n and R₅ are as defined earlier).

The term “alkenyl,” unless otherwise specified, refers to a monoradical of a branched or unbranched unsaturated hydrocarbon group preferably having from 2 to 20 carbon atoms with cis or trans geometry. Particular alkenyl groups include ethenyl or vinyl, 1-propylene or allyl, iso-propylene, bicyclo[2.2.1]heptene, and the like. In the event that alkenyl is attached to the heteroatom, the double bond cannot be alpha to the heteroatom. Alkenyl may further be substituted with one or more substituents selected from alkyl, alkynyl, alkoxy, cycloalkyl, acyl, acylamino, acyloxy, —NRₓR_{y}, —C(═O)NRₓR_{y}, —OC(═O)NRₓR_{y} (wherein Rₓ and R_{y} are as defined earlier), alkoxycarbonylamino, azido, cyano, halogen, hydroxy, oxo, thiocarbonyl, carboxy, —COOR₆ (wherein R₆ is as defined earlier), arylthio, thiol, alkylthio, aryl, alkaryl, aryloxy, heterocyclyl, heteroaryl, heterocyclyl alkyl, heteroaryl alkyl, aminosulfonyl, aminocarbonylamino, alkoxyamino, nitro, —S(O)ₙR₅ (wherein R₅ is as defined earlier). Unless otherwise constrained by the definition, all substituents may optionally be further substituted by 1-3 substituents chosen from alkyl, carboxy, —COOR₆ (wherein R₆ is as defined earlier), hydroxy, alkoxy, halogen, —CF₃, cyano, —NRₓR_{y}, —C(═O)NRₓR_{y}, —OC(═O)NRₓR_{y} (wherein Rₓ and R_{y} are as defined earlier) and —S(O)ₙR₅ (where R₅ and n are as defined earlier).

The term “alkynyl,” unless otherwise specified, refers to a monoradical of an unsaturated hydrocarbon, preferably having from 2 to 20 carbon atoms. Particular alkynyl groups include ethynyl, propargyl or propynyl, and the like. In the event that alkynyl is attached to the heteroatom, the triple bond cannot be alpha to the heteroatom. Alkynyl may further be substituted with one or more substituents selected from alkyl, alkenyl, alkoxy, cycloalkyl, acyl, acylamino, acyloxy, alkoxycarbonylamino, azido, cyano, halogen, hydroxy, oxo, thiocarbonyl, carboxy, —COOR₆ (wherein R₆ is as defined earlier), arylthio, thiol, alkylthio, aryl, alkaryl, aryloxy, aminosulfonyl, aminocarbonylamino, nitro, heterocyclyl, heteroaryl, heterocyclylalkyl, heteroarylalkyl, —NRₓR_{y},—C(═O)NRₓR_{y}, —OC(═O)NRₓR_{y} (wherein Rₓ and R_{y} are as defined earlier), —S(O)ₙR₅ (wherein R₅ is as defined earlier). Unless otherwise constrained by the definition, all substituents may optionally be further substituted by 1-3 substituents chosen from alkyl, carboxy, —COOR₆ (wherein R₆ is as defined earlier), hydroxy, alkoxy, halogen, —CF₃, —NRₓR_{y}, —C(═O)NRₓR_{y}, —OC(═O)NRₓR_{y} (wherein Rₓ and R_{y} are as defined earlier), cyano, and —S(O)ₙR₅ (where R₅ and n are as defined earlier).

The term “cycloalkyl” refers to cyclic alkyl groups of from 3 to 20 carbon atoms having a single cyclic ring or multiple condensed rings, which may optionally contain one or more olefinic bonds, unless otherwise constrained by the definition. Such cycloalkyl groups include, by way of example, single ring structures such as cyclopropyl, cyclobutyl, cyclooctyl, cyclopentenyl, and the like, or multiple ring structures such as adamantanyl, and bicyclo (2.2.1) heptane, or cyclic alkyl groups to which is fused an aryl group, for example indane, and the like. Spiro groups are also contemplated. Cycloalkyl may further be substituted with one or more substituents selected from alkyl, alkenyl, alkynyl, alkoxy, cycloalkyl, acyl, acylamino, acyloxy, alkoxycarbonylamino, azido, cyano, halogen, hydroxy, oxo, thiocarbonyl, carboxy, —COOR₆ (wherein R₆ is as defined earlier), arylthio, thiol, alkylthio, aryl, aralkyl, aryloxy, aminosulfonyl, —NRₓR_{y}, —NHC(═O)NRₓR_{y}, —NHC(═O)Rₓ, —C(═O)NRₓR_{y}, —OC(═O)NRₓR_{y} (wherein Rₓ and R_{y} are as defined earlier), nitro, heterocyclyl, heteroaryl, heterocyclylalkyl, heteroarylalkyl, —S(O)ₙR₅ (wherein R₅ and n are as defined earlier). Unless otherwise constrained by the definition, all substituents may optionally be further substituted by 1-3 substituents chosen from alkyl, carboxy, hydroxy, alkoxy, halogen, CF₃, —NRₓR_{y}, —C(═O)NRₓR_{y}, —NHC(═O)NRₓR_{y}, —OC(═O)NRₓR_{y} (wherein Rₓ and R_{y} are as defined earlier), cyano and —S(O)ₙR₅ (where R₅ and n are as defined earlier).

The term “alkoxy” denotes the group O-alkyl wherein alkyl is the same as defined above.

The term “alkaryl” refers to aryl linked through alkyl (wherein alkyl is the same as defined above) portion and the said alkyl portion contains carbon atoms from 1-6 and aryl is as defined below.

The term “aryl” herein refers to a carbocyclic aromatic group, (for example, phenyl, biphenyl or naphthyl ring and the like optionally substituted with 1 to 3 substituents selected from halogen (F, Cl, Br, I), hydroxy, alkyl, alkenyl, alkynyl, cycloalkyl, alkoxy, acyl, aryloxy, cyano, nitro, —NRₓR_{y}, —C(═O)NRₓR_{y}, —OC(═O)NRₓR_{y} (wherein Rₓ and R_{y} are as defined earlier), carboxy, —COOR₆ (wherein R₆ is as defined earlier), heterocyclyl, heteroaryl, heterocyclylalkyl, heteroarylalkyl or amino carbonylamino.

The term “carboxy,” as defined herein, refers to —C(═O)OH.

The term “heteroaryl,” unless otherwise specified, refers to monocyclic aromatic ring structure containing 5 or 6 ring atoms, a bicyclic or a tricyclic aromatic group having 8 to 10 ring atoms, with one or more heteroatom(s) (N, O or S) optionally substituted with 1 to 3 substituent(s) selected from halogen (F, Cl, Br, I), hydroxy, alkyl, alkenyl, alkynyl, cycloalkyl, acyl, carboxy, —COOR₆ (wherein R₆ is as defined earlier), aryl, alkoxy, alkaryl, cyano, nitro, amino carbonyl amino, —NRₓR_{y}, —C(═O)NRₓR_{y} and —OC(═O)NRₓR_{y} (wherein Rₓ and R_{y} are as defined earlier). Examples of heteroaryl groups include, for example, pyridinyl, pyridazinyl, pyrimidinyl, pyrrolyl, oxazolyl, thiazolyl, thienyl, isoxazolyl, triazinyl, furanyl, benzofuranyl, indolyl, benzothiazolyl, xanthene, benzoxazolyl, and the like.

The term “heterocyclyl,” unless otherwise specified, refers to a non-aromatic monocyclic, bicyclic or tricyclic cycloalkyl group having 5 to 10 atoms in which 1 to 3 carbon atoms in a ring are replaced by heteroatoms (O, S or N), and are optionally benzofused or fused heteroaryl of 5-6 ring members and the heterocyclyl group is optionally substituted wherein the substituents are selected from halogen (F, Cl, Br, I), hydroxy, alkyl, alkenyl, alkynyl, cycloalkyl, acyl, aryl, alkoxy, alkaryl, cyano, nitro, oxo, carboxy, —COOR₆ (wherein R₆ is as defined earlier), aminocarbonylamino, —C(═O)NRₓR_{y}, —OC(═O)NRₓR_{y} (wherein Rₓ and R_{y} are as defined earlier). Examples of heterocyclyl groups include, for example, tetrahydrofuranyl, dihydrofuranyl, dihydropyridinyl, piperidinyl, piperazinyl, dihydrobenzofuryl, azabicyclohexyl, dihydroindolyl, and the like.

“Heteroarylalkyl” refers to heteroaryl (wherein heteroaryl is as defined earlier) linked through alkyl (wherein alkyl is as defined above) portion and the alkyl portion contains from 1-6 carbon atoms.

“Heterocyclylalkyl” refers to heterocyclyl (wherein heterocyclyl is as defined earlier) linked through alkyl (wherein alkyl is the same as defined above) portion and the alkyl portion contains from 1-6 carbon atoms.

“Acyl” refers to —C(═O)R″ wherein R″ is selected from hydrogen, alkyl, cycloalkyl, aryl, aralkyl, hydroxy, alkoxy, heteroaryl, heterocyclyl, heteroarylalkyl or heterocyclylalkyl.

The term “protecting groups” refers to moieties which have the property of preventing specific chemical reaction at a site on the molecule undergoing chemical modification intended to be left unaffected by the particular chemical modification. Also, the term protecting group, unless otherwise specified, may be used with groups such as hydroxy, amino, carboxy and example of such groups are found in T. W. Greene and P. G. M. Wuts, “Protective Groups in Organic Synthesis”, 2^{nd} ed., John Wiley and Sons, New York, N.Y. The species of the carboxylic protecting groups, amino protecting groups or hydroxy protecting groups employed is not critical, so long as the derivatised moiety/moieties is/are stable to conditions of subsequent reactions and can be removed at the appropriate point without disrupting the remainder of the molecule.

In accordance with a second aspect, there is provided a method for treatment or prophylaxis of an animal or a human suffering from a disease or disorder of the respiratory, urinary and gastrointestinal systems, wherein the disease or disorder is mediated through muscarinic receptors. The method includes administration of at least one compound having the structure of Formula I.

In accordance with a third aspect, there is provided a method for treatment or prophylaxis of an animal or a human suffering from a disease or disorder of the respiratory system such as bronchial asthma, chronic obstructive pulmonary disorders (COPD), pulmonary fibrosis, and the like; urinary system which induce such urinary disorders as urinary incontinence, lower urinary tract symptoms (LUTS), etc.; and gastrointestinal system such as irritable bowel syndrome, obesity, diabetes and gastrointestinal hyperkinesis with compounds as described above, wherein the disease or disorder is associated with muscarinic receptors.

In accordance with a fourth aspect, there are provided processes for preparing the compounds as described above.

The compounds described herein exhibit significant potency in terms of their activity, as determined by in vitro receptor binding assays. Pharmaceutical compositions for the possible treatment for the disease or disorders associated with muscarinic receptors are provided. In addition, the compounds can be administered orally or parenterally.

### DETAILED DESCRIPTION OF THE INVENTION

The compounds disclosed herein may be prepared by methods represented by the reaction sequences, for example, as generally shown in Schemes I and II:

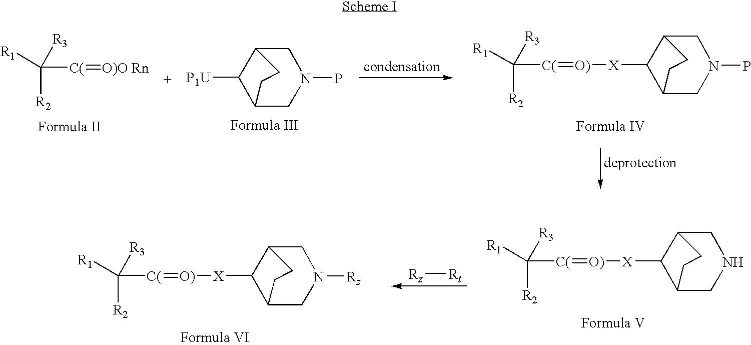

The compounds of Formulae V and VI may be prepared, for example, by the reaction sequence as shown in Scheme I. The preparation comprises condensing a compound of Formula II (wherein Rₙ is hydrogen or alkyl and R₁, R₂ and R₃ are as defined earlier) with a compound of Formula III [wherein U is —O or —O(CH₂)ₙ and P₁ is hydrogen, -mesyl, -tosyl, -triflyl or U is —S or —N(CH₂)n and P₁ is hydrogen; n=1-5, P is a protecting group, for example, aralkyl, —C(═O)Oaralkyl, —C(═O)OC(CH₃)₃, —C(═O)OC(CH₃)₂CHBr₂ or —C(═O)OC(CH₃)₂CCl₃] to give a compound of Formula IV, which undergoes deprotection to give a compound of Formula V, which is reacted with a compound of Formula R_{z}—Rₜ to give a compound of Formula VI [(wherein R_{z} is alkyl or acyl and Rₜ is hal is halogen (F, Cl, Br, I) or —CHO].

The condensation of a compound of Formula II with a compound of Formula III (when U is —O or —S and Rₙ is alkyl) can be carried out in an organic solvent (for example, toluene, heptane or xylene) in the presence of a base (for example, sodium hydride or sodium methoxide) to give a compound of Formula IV. Alternatively, condensation of a compound of Formula II with a compound of Formula III (when U is —O or —S and Rₙ is hydrogen) can be carried out in an organic solvent, for example, dimethylformamide, tetrahydrofuran in the presence of carbonyldiimidazole and a base such as sodium hydride, triethylamine, N-ethyldiisopropylamine or pyridine.

The condensation of a compound of Formula II with a compound of Formula III (when U is —NCH₂ and Rₙ is hydrogen) can be carried out in an organic solvent (for example, dimethylformamide, chloroform, tetrahydrofuran, diethyl ether or dioxane) in the presence of a base (for example, N-methylmorpholine, triethylamine, diisopropylethylamine or pyridine) with a condensing agent (for example, 1-(3-dimethylaminopropyl)-3-ethylcarbodiimide hydrochloride (EDC.HCl) or dicyclohexylcarbodiimide (DCC)) to give a compound of Formula IV.

The condensation of a compound of Formula II with a compound of Formula III (when P₁ is the same as defined earlier, U is —OCH₂ and Rₙ is hydrogen) can be carried out in an organic solvent (for example, toluene, heptane or xylene) in the presence of a base (for example, 1,8-diazabicyclo[5.4.0]undecen-7-ene or 1,4-diazabicyclo[2.2.2]octane) to give a compound of Formula IV.

The deprotection (wherein P can be aralkyl or C(═O)Oaralkyl) of a compound of Formula IV to give a compound of Formula V can be carried out in an organic solvent (for example, ethylacetate, methanol, ethanol, propanol or isopropylalcohol) in the presence of a deprotecting agent (for example, palladium on carbon in presence of hydrogen gas or palladium on carbon with a source of hydrogen gas (for example, ammonium formate, cyclohexene or formic acid)).

The deprotection (wherein P can be —C(═O)Oaralkyl) of a compound of Formula IV to give a compound of Formula V can be carried out with alkaline (for example, potassium hydroxide, sodium hydroxide or lithium hydroxide) solution of an alcohol (for example, methanol, ethanol propanol, diethylether or isopropylalcohol).

The deprotection (when P is —C(═O)OC(CH₃)₃ or —C(═O)OC(CH₃)₂CHBr₂) of a compound of Formula IV to give a compound of Formula V can be carried out in an acidic solution of an alcohol (for example, hydrochloric acid solution of methanol, ethanol, propanol, isopropylalcohol, ethylacetate or ether) or trifluoroacetic acid neat or in dichloromethane.

The deprotection (wherein P can be —C(═O)OC(CH₃)₂CCl₃) of a compound of Formula IV to give a compound of Formula V can be carried out by a supernucleophile, such as, for example, lithium cobalt (I) phthalocyanine, zinc and acetic acid or cobalt phthalocyanine.

The compound of Formula V can undergo N-derivatization with a compound of Formula Rz—Rₜ (when Rₜ is hal and R_{z} is the same as defined earlier) to give a compound of Formula VI in an organic solvent (for example, acetonitrile, dichloromethane, chloroform or carbon tetrachloride) in the presence of a base (for example, potassium carbonate, sodium carbonate or sodium bicarbonate).

The compound of Formula V can undergo reductive amination with a compound of Formula Rz—Rₜ (wherein Rₜ is —CHO and R_{z} is the same as defined earlier) to give a compound of Formula VI in an organic solvent (for example, acetonitrile or dichloromethane) with formaldehyde in the presence of reducing agent (for example, sodium cyanoborohydride or sodium triacetoxy borohydride).

Particular illustrative compounds are mentioned below:

3-Benzyl-3-azabicyclo[3.2.1]oct-8-yl cyclopentyl(hydroxy)phenylacetate (Compound No. 1),
3-azabicyclo[3.2.1]oct-8-yl cyclopentyl(hydroxy)phenylacetate (Compound No. 2),
Hydrochloride salt of 3-azabicyclo[3.2.1]oct-8-yl cyclopentyl(hydroxy)phenylacetate (Compound No. 3),
3-[2-(1,3-benzodioxol-5-yl)ethyl]-3-azabicyclo[3.2.1]oct-8-yl cyclopentyl(hydroxy) phenylacetate (Compound No. 4),
3-Benzyl-3-azabicyclo[3.2.1]oct-8-yl hydroxy(diphenyl)acetate (Compound No. 5),
3-Azabicyclo[3.2.1]oct-8-yl hydroxy(diphenyl)acetate (Compound No. 6),
3-Benzyl-3-azabicyclo[3.2.1]oct-8-yl cyclohexyl(hydroxy)phenylacetate (Compound No. 7),
Hydrochloride salt of 3-Azabicyclo[3.2.1]oct-8-yl hydroxy(diphenyl)acetate (Compound No. 11),
3-Benzyl-3-azabicyclo[3.2.1]oct-8-yl cyclopentyl(hydroxy)(4-methylphenyl)acetate (Compound No. 12),
3-Benzyl-3-azabicyclo[3.2.1]oct-8-yl cyclopentyl(hydroxy)2-thienylacetate (Compound No. 13),
3-Azabicyclo[3.2.1]oct-8-yl cyclopentyl(hydroxy)(4-methylphenyl)acetate (Compound No. 14),
N-(3-Azabicyclo[3.2.1]oct-8-ylmethyl)-2-hydroxy-2,2-diphenylacetamide (Compound No. 15),
N-3-benzyl-3-azabicyclo[3.2.1]oct-8-yl]methyl}-2-hydroxy-2,2-diphenylacetamide (Compound No. 16),
3-Benzyl-3-azabicyclo[3.2.1]oct-8-yl hydroxy(phenyl)2-thienylacetate (Compound No. 17),
3-Benzyl-3-azabicyclo[3.2.1]oct-8-yl cyclopentyl(4-fluorophenyl)hydroxyacetate (Compound No. 18),
N-[(3-benzyl-3-azabicyclo[3.2.1]oct-8-yl)methyl]-2-cyclopentyl-2-hydroxy-2-phenylacetamide (Compound No. 19),
N-[(3-benzyl-3-azabicyclo[3.2.1]oct-8-yl)methyl]-2-cyclopentyl-2-hydroxy-2-(2-thienyl)acetamide (Compound No. 20),
N-[(3-benzyl-3-azabicyclo[3.2.1]oct-8-yl)methyl]-2-hydroxy-2-phenyl-2-(2-thienyl)acetamide (Compound No. 21),
3-Benzyl-3-azabicyclo[3.2.1]oct-8-yl 2-hydroxy-3-methyl-2-phenylbutanoate (Compound No. 22),
N-[(3-benzyl-3-azabicyclo[3.2.1]oct-8-yl)methyl]-3-hydroxy-2-phenylpropanamide (Compound No. 23),
(3-Benzyl-3-azabicyclo[3.2.1]oct-8-yl)methyl cyclopentyl(hydroxy)phenylacetate (Compound No. 24),
3-Benzyl-3-azabicyclo[3.2.1]oct-8-yl 2-(4-fluorophenyl)-2-hydroxy-3-methylbutanoate Compound No. 25),
N-(3-Azabicyclo[3.2.1]oct-8-ylmethyl)-2-cyclopentyl-2-hydroxy-2-phenylacetamide (Compound No. 26),
3-Azabicyclo[3.2.1]oct-8-yl 2-hydroxy-3-methyl-2-phenylbutanoate (Compound No. 27),
N-(3-azabicyclo[3.2.1]oct-8-ylmethyl)-2-hydroxy-2-phenyl-2-(2-thienyl)acetamide (Compound No. 28),
N-(3-azabicyclo[3.2.1]oct-8-ylmethyl)-2-cyclopentyl-2-hydroxy-2-(2-thienyl)acetamide (Compound No. 29),
(3-Benzyl-3-azabicyclo[3.2.1]oct-8-yl)methyl cyclopentyl(hydroxy)2-thienylacetate (Compound No. 30),
(3-Benzyl-3-azabicyclo[3.2.1]oct-8-yl)methyl hydroxy(phenyl)2-thienylacetate (Compound No. 31),
(3-Benzyl-3-azabicyclo[3.2.1]oct-8-yl)methyl hydroxy(diphenyl)acetate (Compound No. 32),
(3-Benzyl-3-azabicyclo[3.2.1]oct-8-yl)methyl hydroxy[bis(3-methylphenyl)]acetate (Compound No. 33),
(3-Benzyl-3-azabicyclo[3.2.1]oct-8-yl)methyl 2,2-diphenylpropanoate (Compound No. 34),
3-Azabicyclo[3.2.1]oct-8-ylmethyl hydroxy(diphenyl)acetate (Compound No. 35),
Tartarate salt of 3-azabicyclo[3.2.1]oct-8-ylmethyl hydroxy[bis(3-methylphenyl)]acetate (Compound No. 36),
3-Azabicyclo[3.2.1]oct-8-ylmethyl 2,2-diphenylpropanoate (Compound No. 37),
(3-benzyl-3-azabicyclo[3.2.1]oct-8-yl)methyl methoxy(diphenyl)acetate (Compound No. 38),
N-[(3-benzyl-3-azabicyclo[3.2.1]oct-8-yl)methyl]-2-methoxy-2,2-diphenylacetamide (Compound No. 39),
3-(4-Methylpent-3-en-1-yl)-3-azabicyclo[3.2.1]oct-8-yl 2-hydroxy-3-methyl-2-phenylbutanoate (Compound No. 40),
2-Cyclopentyl-2-hydroxy-N-{[3-(4-methylpent-3-en-1-yl)-3-azabicyclo[3.2.1]oct-8-yl]methyl}-2-(2-thienyl)acetamide (Compound No. 41),
[3-(4-Methylpent-3-en-1-yl)-3-azabicyclo [3.2.1]oct-8-yl]methyl 2,2-diphenylpropanoate (Compound No. 42),
(4E, 4Z)-N-[(3-benzyl-3-azabicyclo[3.2.1]oct-8-yl)methyl]-2-hydroxy-2-phenylhex-4-enamide (Compound No. 43),
N-[(3-Benzyl-3-azabicyclo[3.2.1]oct-8-yl)methyl]-2-hydroxy-2,2-bis(3-methylphenyl)acetamide (Compound No. 44),
3-Azabicyclo[3.2.1]oct-8-ylmethyl methoxy(diphenyl)acetate (Compound No. 45),
2-(Allyloxy)-N-[(3-benzyl-3-azabicyclo[3.2.1]oct-8-yl)methyl]-2,2-diphenylacetamide (Compound No. 46),
N-[(3-Benzyl-3-azabicyclo[3.2.1]oct-8-yl)methyl]-2,2-diphenyl-2-propoxyacetamide (Compound No. 47),
(3-Benzyl-3-azabicyclo[3.2.1]oct-8-yl)methyl hydroxy(phenyl)pyridin-3-ylacetate (Compound No. 48),
N-[(3-Benzyl-3-azabicyclo[3.2.1]oct-8-yl)methyl]-2-((1R)-3,3-difluorocyclopentyl)-2-hydroxy-2-phenylacetamide (Compound No. 49),
N-[(3-Benzyl-3-azabicyclo[3.2.1]oct-8-yl)methyl]-2-[(1S)-3,3-difluorocyclopentyl]-2-hydroxy-2-phenylacetamide (Compound No. 50),
N-(3-Azabicyclo[3.2.1]oct-8-ylmethyl)-2-[(1S)-3,3-difluorocyclopentyl]-2-hydroxy-2-phenylacetamide (Compound No. 51),
N-(3-Azabicyclo[3.2.1]oct-8-ylmethyl)-2-[(1R)-3,3-difluorocyclopentyl]-2-hydroxy-2-phenylacetamide (Compound No. 52),
Hydrochloride salt of (2R)-N-[(3-benzyl-3-azabicyclo[3.2.1]oct-8-yl)methyl]-2,3-dihydroxy-2-phenylpropanamide (Compound No. 53),
Hydrochloride salt of (2S)-N-[(3-benzyl-3-azabicyclo[3.2.1]oct-8-yl)methyl]-2,3-dihydroxy-2-phenylpropanamide (Compound No. 54),
(2S)-N-[(3-benzyl-3-azabicyclo[3.2.1]oct-8-yl)methyl]-2,3-dihydroxy-2-phenylpropanamide (Compound No. 55),
3-Azabicyclo[3.2.1]oct-8-ylmethyl hydroxy[bis(3-methylphenyl)]acetate (Compound No. 56),
Benzyl 8-({[hydroxy(phenyl)2-thienylacetyl]amino}methyl)-3-azabicyclo[3.2.1]octane-3-carboxylate (Compound No. 57),
Benzyl 8-{[cyclopentyl(hydroxy)2-thienylacetyl]oxy}-3-azabicyclo[3.2.1]octane-3-carboxylate (Compound No. 58),
(2R)-N-[(3-benzyl-3-azabicyclo[3.2.1]oct-8-yl)methyl]-2,3-dihydroxy-2-phenylpropanamide (Compound No. 59),

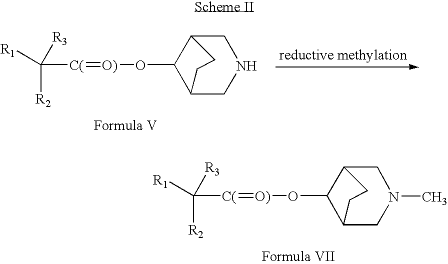

The compound of Formula VII may be prepared, for example, by the reaction sequence as shown in Scheme II. The compound of Formula V undergoes reductive methylation to give a compound of Formula VII.

The reductive methylation of a compound of Formula V can be carried out in an organic solvent (for example, acetonitrile or dichloromethane) with formaldehyde in the presence of reducing agent (for example, sodium cyanoborohydride or sodium triacetoxy borohydride) to give a compound of Formula VII.

One particular illustrative compound is mentioned below:

3-Methyl-3-azabicyclo[3.2.1]oct-8-yl cyclopentyl(hydroxy)phenylacetate (Compound No. 8),

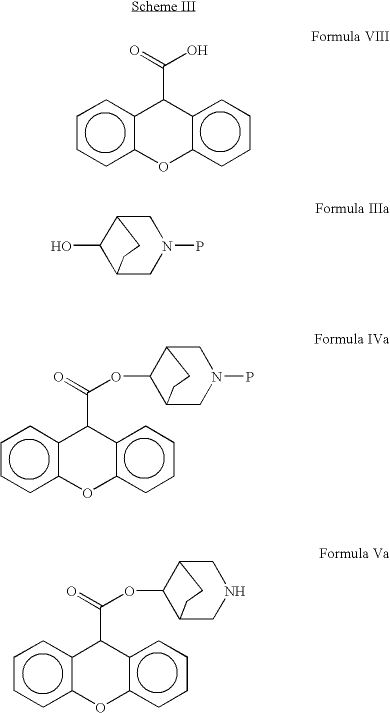

The compound of Formula Va may be prepared, for example, by the reaction sequence as shown in Scheme III. The preparation comprises condensing a compound of Formula VIII with a compound of Formula IIIa to give a compound of Formula IVa (wherein P is the same as defined earlier), which undergoes deprotection to give a compound of Formula Va.

The condensation of a compound of Formula VIII with a compound Formula IIIa can be carried out in an organic solvent (for example, dimethylformamide, tetrahydrofuran, dioxane or diethylether) with carbonyldiimidazole in the presence of a base (for example, sodium hydride or lithium hydride) to give a compound of Formula IV, which can undergo deprotection in an organic solvent (for example, methanol, ethanol, propanol or isopropylalcohol) in the presence of a deprotecting agent (for example, palladium on carbon in the presence of hydrogen gas or palladium on carbon in ammonium formate solution) to give a compound of Formula Va.

Alternatively, carbonyldi-1,2,3-benzotriazole or carbonyldi-1,2-4-triazole can also be used in place of carbonyldiimidazole. Also, the condensation of a compound of Formula VIII with a compound of Formula IIIa can be done by using chloroformates (for example, p-nitrophenyl chloroformate or phenylchloroformate) or condensing agents (for example, 1-(3-dimethylaminopropyl)-3-ethylcarbodiimide hydrochloride or dicyclohexyl carbodiimide.

Particular illustrative compounds are mentioned below:
3-Azabicyclo[3.2.1]oct-8-yl 9H-xanthene-9-carboxylate (Compound No. 9),
3-Benzyl-3-azabicyclo[3.2.1]oct-8-yl 9H-xanthene-9-carboxylate (Compound No. 10),

In the above schemes, where specific bases, condensing agents, protecting groups, deprotecting agents, solvents, catalysts, temperatures, etc. are mentioned, it is to be understood that other bases, condensing agents, protecting groups, deprotecting agents, solvents, catalysts, temperatures, etc. known to those skilled in the art may be used. Similarly, the reaction temperature and duration may be adjusted according to the desired needs.

Suitable salts of the compounds represented by the Formula I were prepared so as to solubilize the compound in aqueous medium for biological evaluations, as well as to be compatible with various dosage formulations and also to aid in the bioavailability of the compounds. Examples of such salts include pharmacologically acceptable salts such as inorganic acid salts (for example, hydrochloride, hydrobromide, sulphate, nitrate and phosphate), organic acid salts (for example, acetate, tartarate, citrate, fumarate, maleate, tolounesulphonate and methanesulphonate). When carboxyl groups are included in the Formula I as substituents, they may be present in the form of an alkaline or alkali metal salt (for example, sodium, potassium, calcium, magnesium, and the like). These salts may be prepared by various techniques, such as treating the compound with an equivalent amount of inorganic or organic, acid or base in a suitable solvent.

TABLE I

| Com- | |
|---|---|
| pound | |
| No. | Structure |
| 1. | 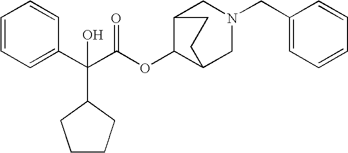 |
| 2. | 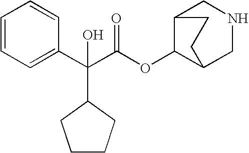 |
| 3.* | 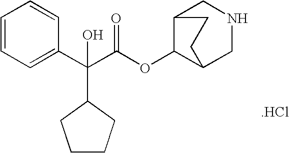 |
| 4. | 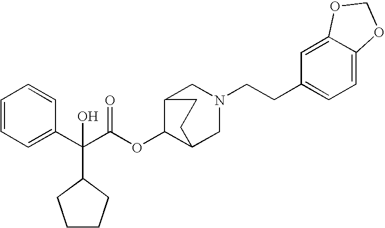 |
| 5. | 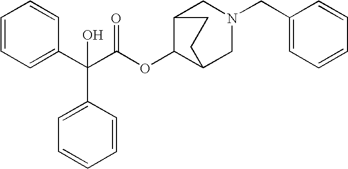 |
| 6. | 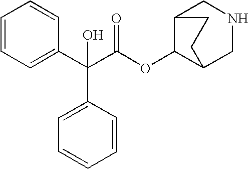 |
| 7. | 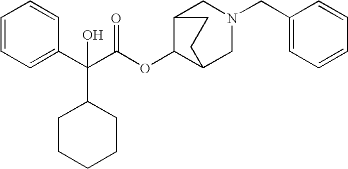 |
| 8. | 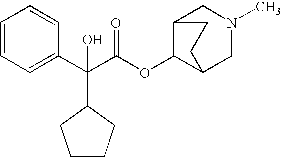 |
| 9. | 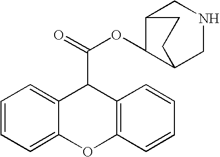 |
| 10. | 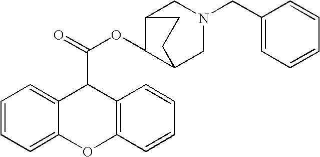 |
| 11.** | 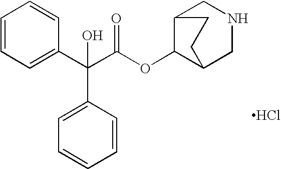 |
| 12. | 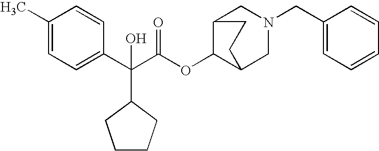 |
| 13. | 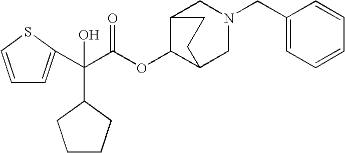 |
| 14. | 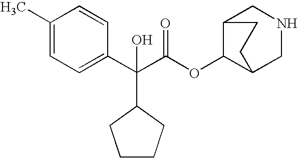 |
| 15. | 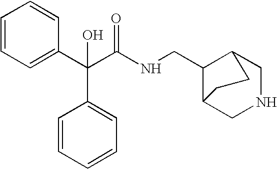 |
| 16. | 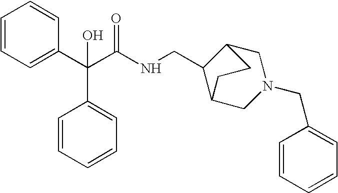 |
| 17. | 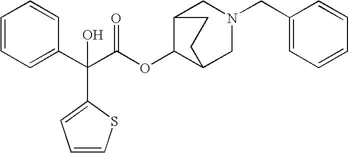 |
| 18. | 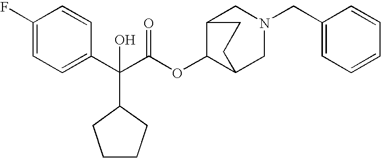 |
| 19. | 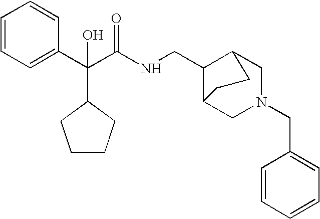 |
| 20. | 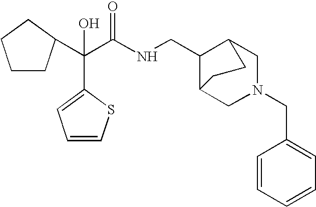 |
| 21. | 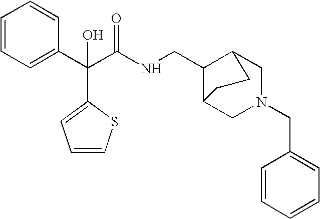 |
| 22. | 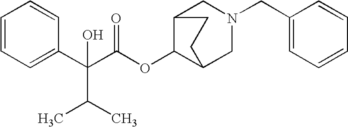 |
| 23. | 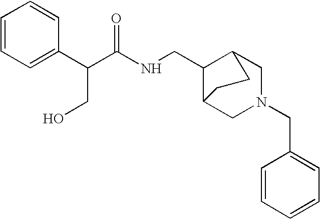 |
| 24. | 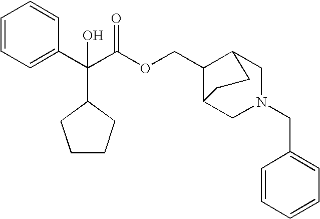 |
| 25. | 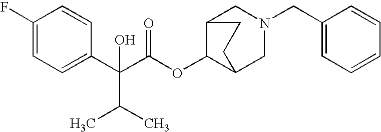 |
| 26. | 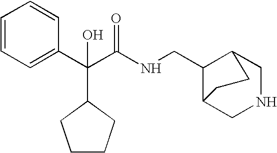 |
| 27. | 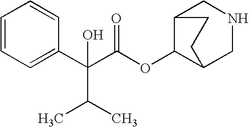 |
| 28. | 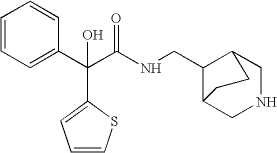 |
| 29. | 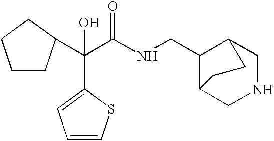 |
| 30. | 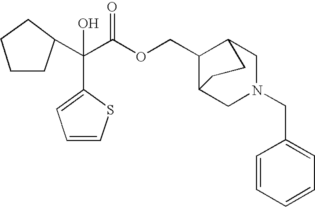 |
| 31. | 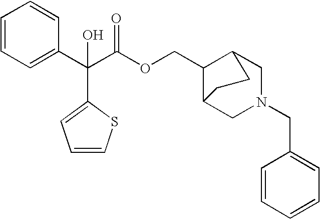 |
| 32. | 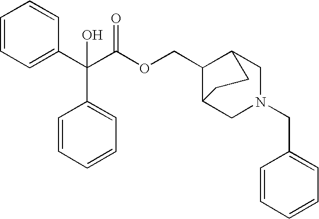 |
| 33. | 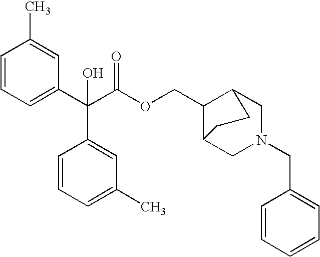 |
| 34. | 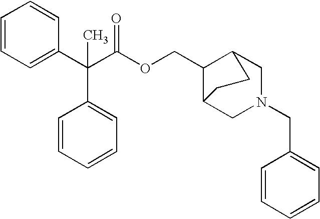 |
| 35. | 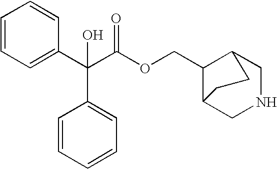 |
| 36. | 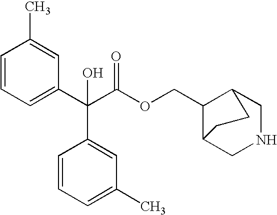 •Tartaric acid |
| 37. | 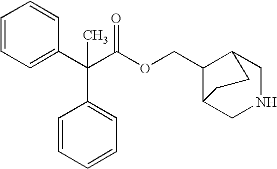 |
| 38. | 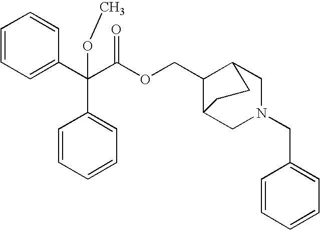 |
| 39. | 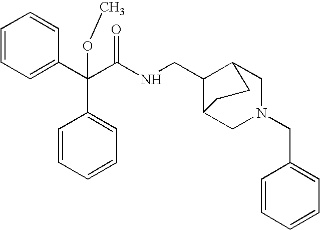 |
| 40. | 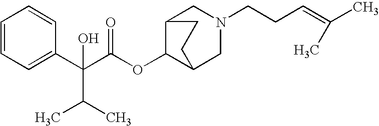 |
| 41. | 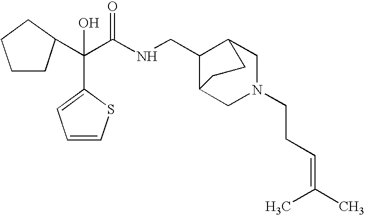 |
| 42. | 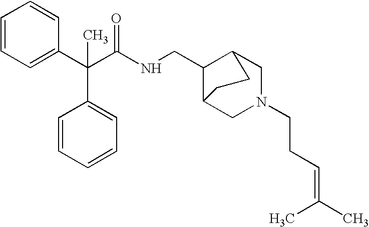 |
| 43. | 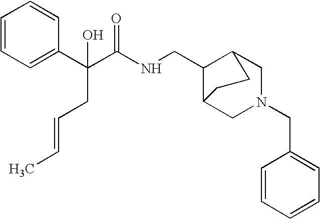 |
| 44. | 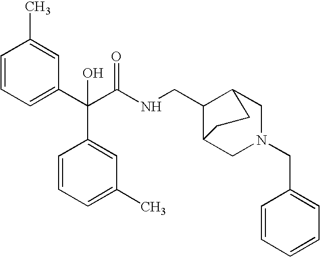 |
| 45. | 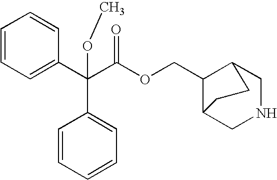 |
| 46. | 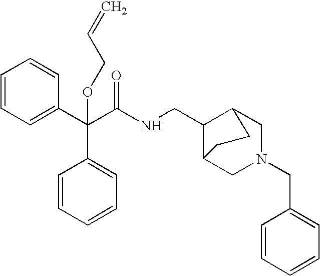 |
| 47. | 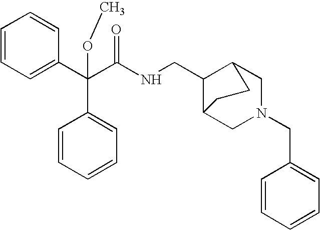 |
| 48. | 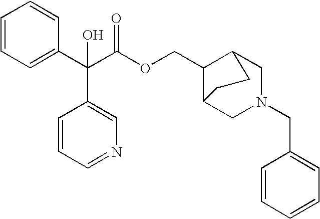 |
| 49. | 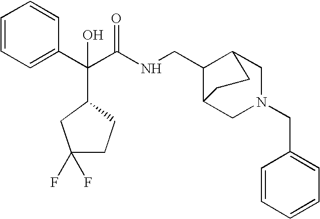 |
| 50.* | 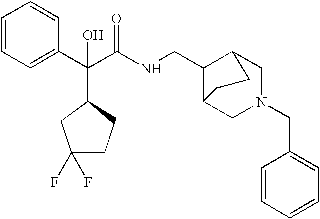 |
| 51.** | 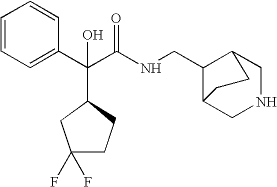 |
| 52. | 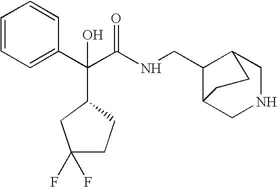 |
| 53. | 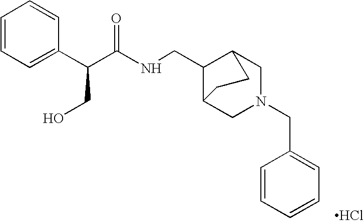 |
| 54. | 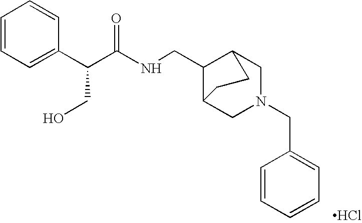 |
| 55. | 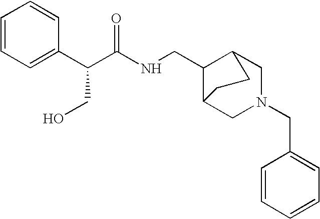 |
| 56. | 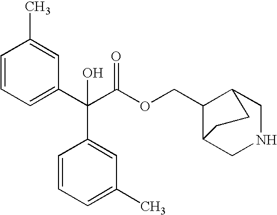 |
| 57. | 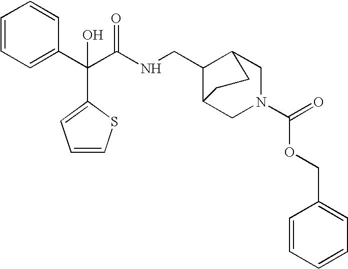 |
| 58. | 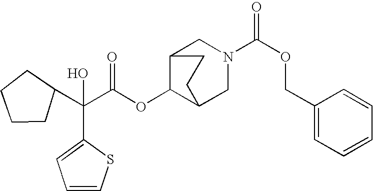 |
| 59. | 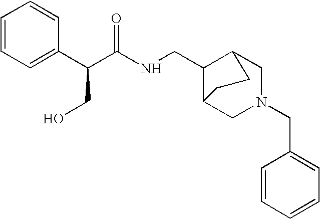 |

*represents hydrochloride salt of Compound No. 2
**represents hydrochloride salt of Compound No. 6

Because of their valuable pharmacological properties, the compounds described herein may be administered to an animal for treatment orally, or by a parenteral route. The pharmaceutical compositions described herein can be produced and administered in dosage units, each unit containing a certain amount of at least one compound described herein and/or at least one physiologically acceptable addition salt thereof. The dosage may be varied over extremely wide limits as the compounds are effective at low dosage levels and relatively free of toxicity. The compounds may be administered in the low micromolar concentration, which is therapeutically effective, and the dosage may be increased as desired up to the maximum dosage tolerated by the patient.

The compounds described herein can be produced and formulated as their enantiomers, diastereomers, N-Oxides, polymorphs, solvates and pharmaceutically acceptable salts, as well as metabolites having the same type of activity. Pharmaceutical compositions comprising the molecules of Formula I or metabolites, enantiomers, diastereomers, N-oxides, polymorphs, solvates or pharmaceutically acceptable salts thereof, in combination with pharmaceutically acceptable carrier and optionally included excipient can also be produced.

The examples mentioned below demonstrate general synthetic procedures, as well as specific preparations of particular compounds. The examples are provided to illustrate the details of the invention and do not limit the scope of the present invention.

### EXAMPLES

Various solvents, such as acetone, methanol, pyridine, ether, tetrahydrofuran, hexanes, and dichloromethane, were dried using various drying reagents according to procedures described in the literature. IR spectra were recorded as nujol mulls or a thin neat film on a Perkin Elmer Paragon instrument, Nuclear Magnetic Resonance (NMR) were recorded on a Varian XL-300 MHz or Bruker 400 MHz instrument using tetramethylsilane as an internal standard.

### Example A

### Synthesis of 3-benzyl-3-azabicyclo[3.2.1]octan-8-ol

To a solution of the compound 3-benzyl-3-azabicyclo-[3.2.1]octan-8-one (5.0 g, 23.3 mmol) in methanol (120 ml) at 0-5° C., was added sodium borohydride (1.1 g, 27.9 mmol) portionwise. The reaction mixture was stirred for 3 hours at the same temperature, and then quenched with water. The solvent was evaporated under reduced pressure and the residual aqueous solution was extracted with dichloromethane. The organic layer was washed with water, brine, dried over anhydrous sodium sulphate and concentrated under reduced pressure to furnish the title compound in a yield of 4.5 g.

¹H NMR (CDCl₃): δ 7.22-7.32 (m, 5H), 3.95-3.98 (m, 1H), 3.53 (s, 2H), 3.29-3.34 (brs, 1H), 2.53-2.63 (m, 2H), 2.44-2.47 (m, 2H), 1.96 (m, 2H), 1.82 (m, 2H), 1.63-1.73 (m, 2H).

### Example B

### Synthesis of ethyl cyclopentyl(hydroxy)(4-methylphenyl)acetate

The title compound was prepared following the procedure as described in J. Am. Chem. Soc., 75, 2654 (1953).

### Example C

### Synthesis of 4E- & 4Z-2-hydroxy-2-phenylhex-4-enoic acid

Step a: 2E- & 2Z-5-[But-2-en-1-yl]-2-tert-butyl-5-phenyl-1,3-dioxolan-4-one

To a solution of lithiumdiisopropylamide (5.36 g, 0.049 mol) in tetrahydrofuran (5 ml) at −78° C. was added a solution of R(−)-2-tert-butyl-5-phenyl-1,3-dioxolan-4-one (10 g, 0.045 mol) in tetrahydrofuran and stirred at same temperature for 30 minutes. To the resulting reaction mixture was added crotyl bromide solution (8.59 g, 0.063 mol) at −78° C. The mixture was allowed to warm to room temperature and again stirred at room temperature for 2 hours followed by quenching with aqueous ammonium chloride. The mixture was then extracted with ethylacetate, washed with water and brine, dried over anhydrous sodium sulphate and concentrated under reduced pressure. The residue thus obtained was purified by column chromatography using 25% ethylacetate in hexane as eluent to furnish the title compound. Yield: 12 g.

Step b: 4 E- & 4Z-2-Hydroxy-2-phenylhex-4-enoic acid

To a solution of the compound obtained from step a above (0.14 g, 0.51 mmol) in methanol (5 ml) was added potassium hydroxide solution (0.28 g in 5 ml water) and stirred for 2 hours. The resulting reaction mixture was concentrated under reduced pressure and the residue thus obtained was diluted with water and extracted with dichloromethane. The aqueous layer was acidified with concentrated hydrochloric acid and extracted with ethylacetate. The organic layer was washed with water and brine, dried over anhydrous sodium sulphate and concentrated under reduced pressure to furnish the title compound. Yield: 80 mg.

¹HNMR (CDCl₃): 7.65-7.61 (m,2H), 7.38-7.26 (m, 3H), 5.74-5.61 (m, 1H), 5.41-5.37 (m, 1H), 3.10-2.96 (m, 1H), 2.89-2.67 (m, 1H), 1.68-1.64 (m, 3H).

### Example D

### Synthesis of (3-benzyl-3-azabicyclo[3.2.1]oct-8-yl)methyl methanesulfonate

Step a: 3-Benzyl-3-aza-bicyclo [3.2.1]octane-8-one

The title compound was prepared following the procedure as described in WO 01/47893 with modifications described below. A solution of the compound cyclopentanone (1.18 mol), paraformaldehyde (3.54 mol) and glacial acetic acid (1.18 mol) in methanol (600 ml) was refluxed for 3 hours. To the resulting reaction mixture was added a solution of benzylamine (0.118 mol) in methanol (200 ml) dropwise and refluxed for 1 hour and subsequently at room temperature for overnight. The mixture was concentrated under reduced pressure and the residue thus obtained was diluted with ethylacetate followed by the addition of sodium metabisulphite (104.6 g). The mixture was stirred for 1 hour. The aqueous layer was separated, cooled under ice and neutralized with sodium carbonate solution. The mixture was extracted with ethylacetate, washed with water and brine, dried over anhydrous sodium sulphate and concentrated under reduced pressure. The residue thus obtained was purified by column chromatography using 5% ethyl acetate in hexane as eluent to furnish the title compound. Yield: 37.5 g.

Step b: Exo 3-benzyl-3-azabicyclo[3.2.1]octane-8-carbaldehyde

To a solution of (methoxymethyl)(triphenyl)phosphonium chloride (11.9 g, 34.88 mmol) in tetrahydrofuran (50 ml) at −50° C. n-butyl lithium (13.7 ml, 34.88 mmol) was added. The reaction mixture was stirred at −25° C. for 30 minutes followed by the addition of a solution of the compound obtained from step a above (5 g, 23.25 mmol) in tetrahydrofuran (10 ml) dropwise at the same temperature. The resulting reaction mixture was stirred at room temperature overnight. The mixture was concentrated under reduced pressure and the residue thus obtained was washed with hexane. The residue was dried under reduced pressure and the residue thus obtained was diluted with tetrahydrofuran followed by the addition of aqueous hydrochloric acid (20%, 30 ml). The reaction mixture was stirred at room temperature for 5 hours. The solvent was evaporated under reduced pressure. The aqueous layer was basified with aqueous potassium hydroxide and extracted with ethylacetate. The organic layer was concentrated under reduced pressure and diluted the residue thus obtained with saturated solution of sodium metabisulphite. The organic layer was separated and neutralized with sodium carbonate. The mixture was extracted with ethylacetate. The organic layer was concentrated under reduced pressure and the residue thus obtained was treated with methanol-water-sodium hydroxide. The solution was stirred at room temperature for 3 days. The reaction mixture was concentrated under reduced pressure and the residue thus obtained was diluted with water. The mixture was extracted with ethylacetate. The organic layer was washed with water and brine, dried under reduced pressure, filtered and concentrated under reduced pressure to furnish the title compound. Yield: 2.2 g.

### ¹H NMR (CDCl₃)δ: 9.62 (s, 1H), 7.32-7.22 (m, 5H), 3.51(s, 2H), 2.79-2.75 (m, 2H), 2.51 (bs, 2H), 2.25 (s, 1H), 2.13-2.05 (m, 2H), 1.79-1.77 (m, 2H), 1.60-1.57 (m, 2H).

Step c: Exo-(3-benzyl-3-azabicyclo [3.2.1]oct-8-yl)methanol

To a solution of the compound obtained from step b above (2.2 g, 9.606 mmol) in methanol cooled in an ice-bath, sodium borohydride (0.545 g, 14.41 mmol) was added and stirred for 2 hours at the same temperature. The mixture was concentrated under reduced pressure and the residue thus obtained was diluted with ethylacetate, washed with water and brine, dried over anhydrous sodium sulphate and concentrated under reduced pressure to furnish the title compound. Yield: 1.8 g.

### ¹ H NMR (CDCl₃)δ: 7.33-7.19 (m, 5H), 3.48 (s, 2H), 3.43-3.41 (d, 2H, J=8 Hz), 2.73-2.69 (m, 2H), 2.09-2.05 (m, 4H), 1.74-1.42 (m, 5H).

Step d: Exo-(3-benzyl-3-azabicyclo[3.2.1]oct-8-yl)methyl methanesulfonate
To a solution of the compound obtained from step c above (0.1 g, 0.4329 mmol) in dichloromethane (5 ml) was added triethylamine (0.1314 g, 1.298 mmol) and 4-dimethylaminopyridine (2 mg, 0.0108 mmol) in dichloromethane followed by the addition of methane sulphonyl chloride (0.126 g, 0.866 mmol) dropwise at 0-30° C. The reaction mixture was stirred at same temperature followed by warming to 25-30° C. with constant stirring for approx. 15 hours. The reaction mixture was diluted with dichloromethane and washed with saturated aqueous solution of sodium bicarbonate. The organic layer was separated, washed with water and brine solution, dried over anhydrous sodium sulphate and concentrated under reduced pressure to yield the title compound. Yield: 0.16 g.

### Example E

### Synthesis of 1-(3-benzyl-3-azabicyclo[3.2.1]oct-8-yl)methanamine

Step a: 3-benzyl-3-azabicyclo[3.2.1]octane-8-carbaldehyde oxime

To a solution of the compound 3-benzyl-3-azabicyclo[3.2.1]octane-8-carbaldehyde (0.25 g, 1.09 mmol) in ethanol (10 ml) was added sodium acetate (0.35 g, 4.25 mmol) and hydroxylamine hydrochloride (0.21 g, 3.065 mmol) and stirred the mixture at room temperature for 2 days. The reaction mixture was concentrated under reduced pressure and the residue thus obtained was diluted with saturated potassium carbonate solution. The mixture was extracted with ethylacetate, washed with water and brine, dried over anhydrous sodium sulphate and concentrated under reduced pressure to furnish the title compound. Yield: 0.2 g.

Step b: Exo-1-(3-benzyl-3-azabicyclo [3.2.1]oct-8-yl)methanamine

To a solution of the compound obtained from step a above (0.19 g, 0.778 mmol) in tetrahydrofuran (10 ml) was added a suspension of lithium aluminium hydride (0.147 g, 3.89 mmol) in tetrahydrofuran (20 ml) and refluxed for 16 hours. The reaction mixture was cooled in an ice bath and quenched with saturated sodium sulphate solution. The mixture was filtered, washed with water and brine dried over anhydrous sodium sulphate and concentrated under reduced pressure to furnish the title compound. Yield: 0.15 g

¹HNMR (CDCl₃): δ 7.34-7.19 (m, 5H), 3.49 (s, 2H), 2.74-2.69 (m, 2H), 2.49-2.46 (m, 2H), 2.09-2.00 (m, 4H ), 1.59-1.25 (m, 5H).

### Example F

### Synthesis of (2R)-3-hydroxy-2-phenylpropanoic acid and (2S)-3-hydroxy-2-phenylpropanoic

A solution of tropic acid (commercially available) (3.06 g) and (−)quinine (6 g) in ethanol (100 ml) was refluxed to dissolve the components and subsequently left at room temperature overnight. The solid thus separated was filtered and was recrystallized from ethanol. The crystallized solid was filtered and recrystallised from ethanol. The salt was neutralized to give (2R)-3-hydroxy-2-phenylpropanoic acid (900 mg). The mother liquor from the first crystallization was concentrated and neutralized to give (2S)-3-hydroxy-2-phenylpropanoic acid: (R)-isomer, SOR(1.1% in MeOH): +50.21 and 90.6% ee; (S)-isomer, SOR (0.7% in MeOH): −50.07 and 88.5% ee

### Example G

### Synthesis of benzyl 8-(aminomethyl)-3-azabicyclo[3.2.1]octane-3-carboxylate

Step a: Tert-butyl [(3-benzyl-3-azabicyclo[3.2.1]oct-8-yl)methyl]carbamate

To a solution of the compound 1-(3-benzyl-3-azabicyclo[3.1.0]hex-6-yl)methanamine (1.5 g, 6.52 mmol) in dichloromethane (30 ml) and ethanol (5 ml) was added triethylamine (7.82 mmol) and tert-butoxycarbonylanhydride (7.17 mmol) and stirred at room temperature overnight. The reaction mixture was concentrated under reduced pressure and the residue thus obtained was diluted with dichloromethane. The organic layer was washed with sodium bicarbonate solution, water and brine, dried over anhydrous sodium sulphate and concentrated under reduced pressure to furnish the title compound. Yield: 2.17 g.

Step b: Tert-butyl (3-azabicyclo[3.2.1]oct-8-ylmethyl)carbamate

To a solution of the compound obtained from step a above (2.17 g) in methanol (50 ml) was added palladium on carbon and stirred at room temperature overnight under hydrogen balloon. The resulting reaction mixture was filtered through celite pad and washed with methanol. The filterate was concentrated under reduced pressure to furnish the title compound. Yield: 1.31 g.

Step c: Benzyl 8-{[(tert-butoxycarbonyl)amino]methyl}-3-azabicyclo[3.2.1]octane-3-carboxylate

To a solution of the compound obtained from step b above (0.0054 mmol) in dichloromethane (20 ml) at 0° C. was added triethylamine (0.016 mmol) and benzylchloroformate (0.0108 mmol). The reaction mixture was stirred at same temperature for 30 minutes and then at room temperature overnight. The mixture was diluted with dichloromethane, washed with sodium bicarbonate solution, waster and brine, dried over anhydrous sodium sulphate and evaporated under reduced pressure. The residue thus obtained was purified by column chromatography to furnish the title compound. Yield: 1.3 g.

Step d: Benzyl 8-{aminomethyl}-3-azabicyclo[3.2.1]octane-3-carboxylate

To a solution of the compound obtained from step c above (1.3 g) in ethanol (10 ml) was added etheral hydrochloric acid (20 ml) and stirred for 3 hours. The reaction mixture was concentrated under reduced pressure and the residue thus obtained was taken in water and basified with aqueous sodium hydroxide solution. The aqueous layer was extracted with ethylacetate, washed with water and brine, dried over anhydrous sodium sulphate and concentrated under reduced pressure to furnish the title compound. Yield: 900 mg. ¹HNMR (CDCl3): δ 7.38-7.26 (5H, m), 5.13 (2H, s), 3.99-3.88 (2H, m), 2.95-2.87 (2H, m), 2.51 (2H, d, 6 Hz), 2.17-1.25 (7H, m); mass (m/z): 275.28 (M⁺+1).

### Example H

### Synthesis of hydroxy(phenyl)pyridin-3-ylacetic acid

Step a: Ethyl hydroxy(phenyl)pyridin-3-ylacetate

A solution of the compound 3-bromo pyridine (5 g, 0.0316 mol) in ether (100 ml) was cooled to −78° C. followed by the addition of n-butyllithium (2.29 g, 0.0348 mol). The mixture was stirred at the same temperature for 1 hour followed by the addition of a solution of ethyl oxo(phenyl)acetate (6.19 g. 0.0348 mol) in ether (20 ml). The reaction mixture was warmed to room temperature and stirred overnight. The reaction mixture was quenched with aqueous ammonium chloride and extracted with ethylacetate. The organic layer was washed with water and brine, dried over anhydrous sodium sulphate, filtered and concentrated under reduced pressure. The residue thus obtained was purified by column chromatography to furnish the title compound. Yield: 4.8 g.

Step b: Hydroxy(phenyl)pyridin-3-ylacetic acid

To a solution of the compound obtained from step a above (2.8 g, 0.010 mol) in ethanol (12 ml) was added dioxane (24 ml), water (4.5 ml) and potassium hydroxide (1.2 g. 0.020 mol) and refluxed for 4 hours. The mixture was concentrated under reduced pressure and the residue thus obtained was diluted with water and extracted with dichloromethane. The aqueous layer was acidified to pH 3. The solid thus separated out was filtered to furnish the title compound as hydrochloride salt. Yield: 2.1 g.

### Example 1

### Synthesis of 3-Benzyl-3-azabicyclo[3.2.1]oct-8-ylcyclopentyl(hydroxy) phenylacetate (Compound No. 1)

A solution of ethyl 2-hydroxy-2-cyclopentylphenyl acetate (1.36 g, 5.82 mmol) [prepared following the procedure described in J. Am. Chem. Soc., 75, 2654 (1953)] and 3-benzyl-3-azabicyclo[3.2.1]octan-8-ol (1.3 g, 5.99 mmol) was refluxed for 30 minutes. To the resulting reaction mixture was added sodium hydride (15 mg, 0.624 mmol) in two portions at an interval of one hour under reflux. The refluxing was continued for 10 hours and the reaction mixture was subsequently cooled to 25-30° C. The reaction mixture was quenched by adding methanol and the solvent was evaporated under reduced pressure. The residue thus obtained was taken in water, extracted with water and brine solution, dried over anhydrous sodium sulphate and concentrated under reduced pressure. The residue thus obtained was purified by column chromatography using ethyl acetate in hexane as eluent. Yield=1.33 g.

IR (DCM): 1720.3 cm⁻¹; ¹H NMR (CDCl₃):δ 7.64-7.67 (m, 2H), 7.30-7.39 (m, 8H), 4.72-4.76 (m, 1HO, 3.40-3.46 (m, 2H), 2.90-2.93 (m, 1H), 2.49-2.51 (m, 2H), 2.19-2.28 (m, 2H), 2.07-2.10 (m, 2H), 1.79-7.82 (m, 2H), 1.36-1.69 (m, 10H); Mass (m/z): 420 (M⁺+1).

Analogues of 3-Benzyl-3-azabicyclo[3.2.1]oct-8-yl cyclopentyl(hydroxy)phenylacetate (Compound No. 1), described below, can be prepared by using appropriate acid in place of 2-hydroxy-2-cyclopentyl-phenyl acetic acid, as applicable in each case.

3-Benzyl-3-azabicyclo[3.2.1]oct-8-yl hydroxy(diphenyl)acetate (Compound No. 5),
m.p: 114-115° C.; IR (KBr): 1711.8 cm⁻¹; ¹H NMR (CDCl₃):δ 7.19-7.48 (m, 15H), 4.88-4.91 (m, 1H), 3.16 (s, 2H), 2.14-2.26 (m, 4H), 1.92-1.95 (m, 2H), 1.72-1.83 (m, 2H), 1.56-1.66 (m, 2H); Mass (m/z): 428 (M⁺+1).
3-Benzyl-3-azabicyclo[3.2.1]oct-8-yl cyclohexyl(hydroxy)phenylacetate (Compound No. 7),
IR (DCM): 1721.2 cm⁻¹; ¹H NMR (CDCl₃):δ 7.64-7.67 (m, 2H), 7.24-7.40 (m, 8H), 4.74-4.77 (m, 1H), 3.48 (s, 2H), 2.51-2.52 (m, 2H), 2.01-2.31 (m, 5H), 1.46-1.82 (m, 14H).; Mass(m/z): 434 (M⁺+1), 416 (M—OH).
3-Benzyl-3-azabicyclo[3.2.1]oct-8-yl cyclopentyl(hydroxy)(4-methylphenyl)acetate (Compound No. 12)
The title compound was prepared following the procedure as described in Example 1 in the presence of a co-solvent toluene.
¹HNMR: (CDCl₃)δ: 7.30-7.09 (m, 9H), 4.79-4.76 (m, 1H), 3.37 (s, 2H), 2.90-2.88 (m, 2H), 2.41-2.22 (m, 11H), 1.81-1.60 (m, 9H); Mass (m/z): 416 (M-17).
3-Benzyl-3-azabicyclo[3.2.1]oct-8-yl cyclopentyl(hydroxy)2-thienylacetate (Compound No. 13)
1H NMR (CDCl₃)δ: 7.32-6.95 (m, 8H), 4.80 (m, 1H), 4.07 (s, 1H), 3.48 (s, 2H), 2.80-2.77 (m, 1H), 2.53-2.50 (m, 2H), 2.37-2.14 (m, 6H), 1.84-1.26 (m, 10H); Mass (m/z): 426 (M⁺+1).
3-Benzyl-3-azabicyclo[3.2.1]oct-8-yl hydroxy(phenyl)2-thienylacetate (Compound No. 17)
¹H NMR (CDCl₃)δ: 7.58 (2H, bs), 7.35 (2H, bs), 7.26 (7H, bs), 7.15 (1H, d, 6 Hz), 7.03 (1H, t, 3 Hz), 4.86 (1H, bs), 4.56 (1H, s), 3.20 (2H, dd, 3 Hz & 12 Hz), 2.36 (1H, bs), 2.2 (4H, bs), 1.84 (3H, bs), 1.26 (2H, s); Mass (m/z): 434 (M⁺+1)

### Example 2

### Synthesis of 3-Benzyl-3-azabicyclo[3.2.1]oct-8-yl cyclopentyl(4-fluorophenyl)hydroxyacetate (Compound No. 18)

To a solution of the 3-benzyl-3-azabicyclo[3.2.1]octan-8-ol (0.501 g, 2.310 g) in dimethylformamide (5 ml) was added sodium hydride (4.201 mmol) and stirred at room temperature for 2 hours. To the resulting reaction mixture was added a solution of cyclopentyl(4-fluorophenyl)hydroxyacetic acid (2.1008 mmol), carbonyldiimidazole (2.52 mmol) in dimethylformamide (5 ml) and stirred for 5 hours. The mixture was quenched with water and extracted with ethylacetate. The organic layer was washed with water and brine, dried over anhydrous sodium sulphate and concentrated under reduced pressure. The residue thus obtained was purified by column chromatography using 3% ethylacetate in hexane as eluent to furnish the title compound. Yield: 400 mg.

¹H NMR (CDCl₃)δ: 7.63 (2H, bs), 7.30 (5H, bs), 7.02 (2H, m), 4.74 (1H, t, 6 Hz), 3.85 (1H, s), 3.46 (2H, dd, J=12 Hz & 3 Hz), 2.86 (1H, bm), 2.50 (2H, d, J=3 Hz), 2.30 (1H, m), 2.20 (1H, s), 2.09 (2H, bs), 1.83-1.26 (12H, m); Mass (m/z): 438 (M⁺+1).
Analogues of 3-Benzyl-3-azabicyclo[3.2.1]oct-8-yl cyclopentyl(4-fluorophenyl)hydroxyacetate (Compound No. 18) described below can be prepared similarily,
3-Benzyl-3-azabicyclo[3.2.1]oct-8-yl 2-hydroxy-3-methyl-2-phenylbutanoate (Compound No. 22)
¹H NMR (CDCl₃)δ: 7.66 (2H, d, 9 Hz), 7.40-7.26 (8H, m), 4.76 (1H, t, J=6 Hz & 3 Hz), 3.75 (1H, s), 3.46 (2H, bs), 2.54 (3H, m), 2.20-2.09 (4H, m), 1.81 (2H, bm), 1.33 (4H, bs), 0.98 (3H, d, 6 Hz), 0.75 (3H, d, 6 Hz); Mass(m/z): 393 (M+1).
3-Benzyl-3-azabicyclo[3.2.1]oct-8-yl 2-(4-fluorophenyl)-2-hydroxy-3-methylbutanoate (Compound No. 25)
¹H NMR (CDCl₃)δ: 7.63 (2H, bs), 7.30-7.25 (5H, bd), 7.05 (2H, t, J=9 Hz), 4.76 (1H, bs), 3.76 (1H, s), 3.47 (2H, bs), 2.51 (3H, bs), 2.30-2.07 (4H, m), 1.83-1.45 (4H, m), 0.97 (3H, d, J=6 Hz), 0.73 (3H, d, J=6 Hz); Mass (m/z): 412 (M⁺+1).

### Example 3

### Synthesis of N-3-benzyl-3-azabicyclo[3.2.1]oct-8-yl]methyl}-2-hydroxy-2,2-diphenylacetamide (Compound No. 16)

To a solution of the compound hydroxy(diphenyl)acetic acid (0.138 g, 0.608 mmol) and 1-(3-benzyl-3-azabicyclo[3.1.0]hex-6-yl)methanamine (0.14 g, 0.608 mmol) in dimethylformamide (10 ml) was added hydroxybenzotriazole (0.904 g, 0.669 mmol) and N-methylmorpholine (0.12 g, 1.21 mmol) at 0° C. The resulting reaction mixture was stirred at 0° C. for 1 hour followed by the addition of 1-(3-dimethylaminopropyl)-3-ethylcarbodiimide hydrochloride (0.116 g, 0.6086 mmol). The reaction mixture was further stirred at 0° C. for 1 hour and then at room temperature overnight. The reaction mixture was poured into sodium bicarbonate solution and extracted with ethylacetate. The ethylacetate layer was washed with water and brine, dried over anhydrous sodium sulphate and concentrated under reduced pressure. The residue thus obtained was purified by column chromatography using 20% ethylacetate in hexane as eluent to furnish the title compound. Yield: 0.1 g.

### ¹H NMR (CDCl₃)δ: 7.43-7.25 (m, 15H), 6.28 (bs, 1H), 3.93 (s, 1H), 3.46 (s, 2H), 3.16-3.11 (m, 2H), 2.69-2.65 (m, 2H), 2.04-2.00 (d, 2H, J=16 Hz), 1.86 (bs, 2H), 1.73-1.41 (m, 5H); Mass(m/z): 441 (M⁺+1).

Analogues of N-3-benzyl-3-azabicyclo[3.2.1]oct-8-yl]methyl}-2-hydroxy-2,2-diphenylacetamide (Compound No. 16) described below were prepared similarily,
N-[(3-benzyl-3-azabicyclo[3.2.1]oct-8-yl)methyl]-2-cyclopentyl-2-hydroxy-2-phenylacetamide (Compound No. 19)
¹H NMR (CDCl₃)δ: 7.61 (2H, d, J=3 Hz), 7.28 (8H, m), 6.38 (1H, m), 3.44 (2H, m), 3.21 (1H, s), 3.03 (3H, m), 2.65 (2H, bs), 2.01-1.81 (4H, m), 1.64 (13H, bm); Mass(m/z): 433 (M⁺+1).
N-[(3-benzyl-3-azabicyclo[3.2.1]oct-8-yl)methyl]-2-cyclopentyl-2-hydroxy-2-(2-thienyl)acetamide (Compound No. 20)
¹H NMR (CDCl₃)δ: 7.30-7.21 (6H,m), 7.07 (1H, bs), 6.96 (1H, bs), 6.31 (1H, bs), 3.72 (1H, bs), 3.45 (2H, bs), 3.09-3.00 (2H, m), 2.82 (1H, bm), 2.67 (2H, bs), 2.01 (2H, bd), 1.85 (2H, bs), 1.64 (13H, bs); Mass (m/z): 439 (M⁺+1).
N-[(3-benzyl-3-azabicyclo[3.2.1]oct-8-yl)methyl]-2-hydroxy-2-phenyl-2-(2-thienyl)acetamide (Compound No. 21)
¹H NMR (CDCl₃)δ: 7.48 (2H, bs), 7.30 (9H, bm), 6.97 (2H, s), 6.26 (1H, bs), 4.29 (1H, bs), 3.46 (2H, s), 3.13 (2H, bs), 2.68 (2H, bs), 2.04 (2H, bs), 1.87 (2H, bs), 1.53 (5H, bm); Mass(m/z): 447 (M+1).
N-[(3-benzyl-3-azabicyclo[3.2.1]oct-8-yl)methyl]-3-hydroxy-2-phenylpropanamide (Compound No. 23)
¹H NMR (CDCl₃)δ: 7.35-7.26 (m, 10H), 5.4 (bs, 1H), 4.14 (m, 1H), 3.77-3.65 (m, 2H), 3.45 (m, 3H), 3.08-3.02 (m, 2H), 2.67-2.64 (m, 2H), 2.01-1.98 (m, 2H), 1.83 (bs, 2H), 1.69-1.55 (m, 5H); Mass (m/z): 379 (M⁺+1).
N-[(3-benzyl-3-azabicyclo[3.2.1]oct-8-yl)methyl]-2-methoxy-2,2-diphenylacetamide (Compound No. 39)
¹H NMR (CDCl₃)δ: 7.45-7.17 (15H, m), 3.48 (2H, s), 3.10 (2H, m), 3.03 (3H, s), 2.68 (2H, bs), 2.06-2.03 (4H, m), 1.28 (5H, m); Mass (m/z): 455 (M⁺+1).
(4E, 4Z)-N-[(3-benzyl-3-azabicyclo[3.2.1]oct-8-yl)methyl]-2-hydroxy-2-phenylhex-4-enamide (Compound No. 43)

### ¹H NMR (CDCl₃)δ: 7.65-7.60 (d, 2H), 7.35-7.26 (m, 8H), 6.77 (s, 1H), 5.71-5.66 (m, 1H), 5.35-5.27 (m, 1H), 3.64 (s, 1H), 3.55 (s, 2H), 3.07-2.65 (m, 8H), 2.12-1.26 (m, 10H).

N-[(3-Benzyl-3-azabicyclo[3.2.1]oct-8-yl)methyl]-2-hydroxy-2,2-bis(3-methylphenyl) acetamide (Compound No. 44)

¹H NMR (CDCl₃)δ: 7.32-7.12 (m, 13H), 3.54 (s, 2H), 3.15-3.11 (m, 2H), 2.8 (m, 2H), 2.3 (s, 6H), 2.18-1.26 (m, 9H); Mass (m/z): 469 (M⁺+1).

2-(Allyloxy)-N-[(3-benzyl-3-azabicyclo[3.2.1]oct-8-yl)methyl]-2,2-diphenylacetamide (Compound No. 46)

¹H NMR (CDCl₃)δ: 7.46-7.25 (m, 15H), 6.01 (m, 1H), 5.3 (d, 1H,J=4 Hz), 5.29 (d, 1H, J=4 Hz), 3.60-3.57 (m, 4H), 3.11-3.07 (m, 2H), 2.04 (m, 2H), 1.90 (bs, 2H), 1.71-1.25 (m, 7H); Mass (m/z): 481 (M⁺+1).

N-[(3-Benzyl-3-azabicyclo[3.2.1]oct-8-yl)methyl]-2,2-diphenyl-2-propoxyacetamide (Compound No. 47)

¹H NMR (CDCl₃)δ: 7.44-7.20 (m, 15H), 3.65 (s, 2H), 3.11-3.08 (m, 2H), 3.02-2.98 (m, 2H), 2.8 (m, 2H), 2.02 (m, 2H), 1.92-1.57 (m, 8H), 0.92 (m, 3H); Mass (m/z): 483 (M⁺+1).

Benzyl 8-({[hydroxy(phenyl)2-thienylacetyl]amino} methyl)-3-azabicyclo[3.2.1]octane-3-carboxylate (Compound No. 57)

¹H NMR (CDCl₃)δ: 7.51-6.98(m,13H), 6.4(s,1H), 5.13(s,2H), 4.17(s,1H), 3.8-4.0(m,2H), 3.15-3.13(m,2H), 2.89-2.80(m,2H), 1.98-1.93(m,2H), 1,81(m,2H), 1.74-1.63(m,3H)

Benzyl 8-{[cyclopentyl(hydroxy)2-thienylacetyl]oxy}-3-azabicyclo[3.2.1]octane-3-carboxylate (Compound No. 58)

¹H NMR (CDCl₃)δ: 7.36-7.33(m,5H), 7.23(d,1H), 7.08(d,1H), 6.97(s,1H),6.47(s,1H),3.91(m,2H), 3.59(m, 1H), 2.89-2.79(m,5H), 1.77-1 .26(m,14H)

### Example 4

### Syntheses of N-[(3-Benzyl-3-azabicyclo[3.2.1]oct-8-yl)methyl]-2R-((1R)3,3-difluorocyclopentyl)-2-hydroxy-2-phenylacetamide (Compound No. 49) and N-[(3-Benzyl-3-azabicyclo[3.2.1 ]oct-8-yl)methyl]-2R-[(1S)-3,3-difluorocyclopentyl]-2-hydroxy-2 phenylacetamide (Compound No. 50)

To a solution of the compound (2R)-(3,3-difluorocyclopentyl)(hydroxy)phenylacetic acid (172.01 mg, 0.671 mmol) and 1-(3-benzyl-3-azabicyclo[3.1.0]hex-6-yl)methanamine (0.739mmol) in dimethylformamide (10 ml) was added hydroxybenzotriazole (0.739 mmol) and N-methylmorpholine (1.34mmol) at 0° C. The resulting reaction mixture was stirred at 0° C. for 1 hour followed by the addition of 1-(3-dimethylaminopropyl)-3-ethylcarbodiimide hydrochloride (0.67mmol). The reaction mixture was further stirred at 0° C. for 1 hour and then at room temperature overnight. The reaction mixture was poured into sodium bicarbonate and extracted with ethylacetate. The ethylacetate layer was washed with water and brine, dried over anhydrous sodium sulphate and concentrated under reduced pressure. The residue thus obtained was purified by column chromatography using 25% ethylacetate in hexane as eluent to furnish the title compounds. Yield: (60 mg, Compound No. 49) and (65 mg, Compound No. 50).

¹H NMR (CDCl₃, Compound No. 49)δ: 7.56 (2H, d, 6 Hz), 7.37 (2H, t, 6 Hz), 7.31-7.22 (6H, m), 6.27 (1H, bs), 3.51 (1H, s), 3.45 (2H, s), 3.31 (1H, m), 2.98 (2H, m), 2.65 (2H, bs), 2.14-1.51 (15H, m); Mass (m/z): 469 (M⁺+1).
¹H NMR (CDCl₃, Compound No. 50)δ: 7.55 (2H, d, 6 Hz), 7.36 (2H, t, 6 Hz), 7.30-7.21 (6H, m), 6.33 (1H, bs), 3.47 (1H, s), 3.44 (2H, s), 3.32 (1H, m), 2.96 (2H, m), 2.64 (2H, bs), 2.13-1.54 (15H, m); Mass(m/z): 469 (M⁺+1).

### Example 5

### Synthesis of (2R)-N-[(3-benzyl-3-azabicyclo[3.2.1]oct-8-yl)methyl]-3-hydroxy-2-phenylpropanamide (Compound No. 59)

The title compound was prepared following the procedure as described for the synthesis of Compound No. 16 by using in (2R)-3-hydroxy-2-phenylpropanoic acid in place of hydroxy(diphenyl)acetic acid.

¹H NMR (CDCl₃) δ: 7.37-7.23(m, 10H), 5.49 (s, 1H), 4.16-4.11 (m,1H), 3.78-3.65 (m, 2H), 3.46 (s, 2H), 3.07-3.02 (m, H), 2.67-2.66 (m, 2H), 2.02-1.99 (m, 2H), 1.83-1.54 (m, 7H).
The following compound was prepared similarily,
(2S)-N-[(3-benzyl-3-azabicyclo[3.2.1]oct-8-yl)methyl]-3-hydroxy-2-phenylpropanamide (Compound No. 55)

### ¹H NMR (CDCl₃) δ: 7.37-7.21 (m, 10H), 5.4 (s, 1H), 4.13-4.11 (m, 1H), 3.67-3.64 (m, 2H), 3.45 (s, 2H), 3.07-3.02(m, 2H), 2.67-2.63(m, 2H), 2.00-1.98 (m, 2H), 1.82(m, 2H), 1.71-1.54(m, 5H)

### Example 6

### Synthesis of (3-Benzyl-3-azabicyclo[3.2.1]oct-8-yl)methyl 2,2-diphenylpropanoate (Compound No. 34)

To a solution of the compound 2,2-diphenylpropanoic acid (1.45 mmol) and 3-benzyl-3-azabicyclo[3.2.1]oct-8-yl methanesulfonate (300 mg, 0.97 mmol) in toluene was added 1,8-diazabicyclo[5.4.0]undec-7-ene (1.94 mmol) and refluxed for 14 hours. The reaction mixture was concentrated under reduced pressure and the residue thus obtained was purified by column chromatography using 5% ethyl acetate in hexane as eluent to furnish the title compound. Yield: 280 mg.

¹H NMR (CDCl₃)δ: 7.42-7.25 (15H, m), 4.25 (1H, bs), 4.05 (2H, d, J=6 Hz), 3.44 (2H, s), 2.62 (2H, bs), 2.10-1.25 (9H, m); Mass (m/z): 442 (M⁺+1).
Analogues of (3-Benzyl-3-azabicyclo[3.2.1]oct-8-yl)methyl 2,2-diphenylpropanoate (Compound No. 34) described below were prepared similarily,
(3-Benzyl-3-azabicyclo[3.2.1]oct-8-yl)methyl cyclopentyl(hydroxy)phenylacetate (Compound No. 24)
¹H NMR (CDCl₃)δ: 7.65-7.63 (d, 2H, J=8 Hz), 7.36-7.22 (m, 8H), 3.99-3.92 (m, 2H), 3.77 (s, 1H), 3.48 (s, 2H), 2.68 (m, 1H), 2.67 (m, 2H), 2.18-1.26 (m, 17H); Mass (m/z): 434 (M⁺+1).
(3-Benzyl-3-azabicyclo[3.2.1]oct-8-yl)methyl cyclopentyl(hydroxy)2-thienylacetate (Compound No. 30)

### ¹H NMR (CDCl₃)δ: 7.31 (5H, s), 7.19 (1H, s), 7.10 (1H, s), 6.95 (1H, d, J=6 Hz), 4.00 (3H, m), 3.48 (2H, s), 2.70 (4H, m), 2.10-0.99 (16H, m); Mass(m/z): 440 (M⁺+1).

(3-Benzyl-3-azabicyclo[3.2.1]oct-8-yl)methyl hydroxy(phenyl)2-thienylacetate (Compound No. 31)
¹H NMR (CDCl₃)δ: 7.50 (2H, d, 6 Hz), 7.31 (9H, bs), 7.09 (1H, d, J=3 Hz), 6.98 (1H, d, J=3 Hz), 4.51 (1H, s), 4.07 (2H, d, J=6 Hz), 3.45 (2H, s), 2.66-2.63 (3H, m), 2.10-1.00 (8H, m); Mass (m/z): 448 (M⁺+1).
(3-Benzyl-3-azabicyclo[3.2.1]oct-8-yl)methyl hydroxy(diphenyl)acetate (Compound No. 32)
1H NMR (CDCl₃)δ: 7.42-7.25 (15H, m), 4.25 (1H, bs), 4.05 (2H, d, J=6 Hz), 3.44 (2H, s), 2.62 (2H, bs), 2.10-1.25 (9H, m); Mass (m/z): 442 (M⁺+1).
(3-Benzyl-3-azabicyclo[3.2.1]oct-8-yl)methyl hydroxy[bis(3-methylphenyl)]acetate (Compound No. 33)
¹H NMR (CDCl₃)δ: 7.30-7.11 (13H, m), 4.20 (1H, s), 4.06 (2H, d, J=6 Hz), 3.45 (2H, s), 2.64 (2H, m), 2.32 (6H, s), 2.0 (2H, d, J=6 Hz), 1.87 (2H, bs), 1.81-1.69 (3H, m) 1.30-1.26 (2H, m); Mass (m/z): 470.1 (M⁺+1).
(3-benzyl-3-azabicyclo[3.2.1]oct-8-yl)methyl methoxy(diphenyl)acetate (Compound No. 38)
¹H NMR (CDCl₃)δ: 7.44-7.20 (15H, m), 3.98 (2H, d, J=6 Hz), 3.43 (2H, s), 3.16 (3H, s),2.58 (2H, m), 1.95 (H, d, J=9 Hz), 1.79-1.28 (7H, m). Mass (m/z): 456 (M⁺+1).
(3-Benzyl-3-azabicyclo[3.2.1]oct-8-yl)methyl hydroxy(phenyl)pyridin-3-ylacetate (Compound No. 48)
¹H NMR (CDCl₃)δ: 8.69 (1H, s), 8.55 (1H, s), 7.77 (1H, d, J=6 Hz), 7.38-7.21 (11H, m), 4.30 (1H, s), 4.09 (2H, d, J=6 Hz), 3.45 (2H, s), 2.64 (2H, dd, J=3 Hz & 3 Hz), 1.99 (2H, d, J=6 Hz), 1.84-0.99 (7H, m); Mass (m/z): 442 (M⁺+1).

### Example 7

### Synthesis of 3-azabicyclo[3.2.1]oct-8-yl cyclopentyl(hydroxy)phenylacetate (Compound No. 2)

To a solution of the Compound No. 1 (1.0 g, 2.39 mmol) in methanol (25 ml), was added palladium on carbon (10%, 42 mg) and ammonium formate (0.75 g) under N₂ atmosphere. The reaction mixture was refluxed for 0.5 hours and subsequently cooled 25-30° C. The reaction mixture was filtered through celite pad, washed with methanol, ethylacetate and water. The combined filtrate was concentrated under reduced pressure. The residue thus obtained was diluted with water and washed with dichloromethane. The pH of the aqueous layer was adjusted to 14 with 10% aqueous sodium hydroxide and extracted with dichloromethane. The organic layer was washed with water and brine solution, dried over anhydrous sodium sulphate and concentrated under reduced pressure to furnish the title compound. Yield=0.51 g;

m.p: 127.0-127.5° C.; IR (KBr): 1734.1 cm⁻¹; ¹H NMR (CDCl₃):δ 7.67-7.69 (m, 2H), 7.28-7.40 (m, 3H), 4.87-4.90 (m, 1H), 3.34 (d, J=12 Hz, 1H), 3.00-3.04 (m, 2H), 2.87-2.91 (m, 1H), 2.73-2.77 (m, 1H), 2.24-2.31 (m, 2H), 1.92-1.98 (m, 4H), 1.39-1.51 (m, 8H); Mass (m/z): 330 (M⁺+1), 312 (M—OH).
Analogues of 3-azabicyclo[3.2.1]oct-8-yl cyclopentyl(hydroxy)phenylacetate (Compound No. 2) described below, can be prepared by deprotecting appropriate N-benzylated amine in place of 3-benzyl-3-azabicyclo[3.2.1]oct-8-yl cyclopentyl(hydroxy)phenylacetate, respectively, as applicable in each case.
3-Azabicyclo[3.2.1]oct-8-yl hydroxy(diphenyl)acetate (Compound No. 6),
m.p: 138.3-138.5° C.; IR (KBr): 1723.1 cm⁻¹; ¹H NMR (CDCl₃):δ 7.34-7.48 (m, 10H), 5.00-5.04 (m, 1H), 2.68-2.72 (m, 2H), 2.27-2.34 (m, 2H), 1.61-1.80 (m, 6H); Mass (m/z): 338 (M⁺+1), 320 (M—OH).
3-azabicyclo[3.2.1]oct-8-ylmethyl 2,2-diphenylpropanoate (Compound No. 37)
¹H NMR (CDCl₃):δ 7.32-7.21 (10H, m), 3.92 (2H, d, J=6 Hz), 2.91 (2H, d, J=9 Hz), 2.75 (2H, d, J=9 Hz), 2.10 (1H, s), 1.99 (3H, s), 1.93 (3H, bs), 1.76 (3H,s); Mass (m/z): 350 (M⁺+1).
3-Azabicyclo[3.2.1]oct-8-yl cyclopentyl(hydroxy)(4-methylphenyl)acetate (Compound No. 14)

### ¹H NMR (CDCl₃)δ: 7.26-7.24 (m, 2H), 7.12-7.10 (m, 2H), 4.81-4.77 (m, 1H), 3.34-3.30 (d, J=11 Hz, 1H), 3.15-3.11 (d, J=12 Hz, 1H), 2.95 (m, 1H), 2.31 (m, 6H), 2.1 (s, 3H), 1.93-1.26 (m, 10H).

N-(3-Azabicyclo[3.2.1]oct-8-ylmethyl)-2-hydroxy-2,2-diphenylacetamide (Compound No. 15)

### 1H NMR (CDCl₃)δ: 7.41-7.26 (m, 10H), 3.16-3.09 (m, 2H), 2.69 (m, 4H), 1.83-1.54 (m, 7H);

### Mass(m/z): 351 (M⁺+1); m.pt.: 85.2° C.

N-(3-Azabicyclo[3.2.1]oct-8-ylmethyl)-2-cyclopentyl-2-hydroxy-2-phenylacetamide (Compound No. 26)
¹H NMR (CDCl₃)δ: 7.60 (2H, d, J=6 Hz), 7.34 (3H, m), 6.46 (1H, bs), 3.05-2.92 (3H, m), 2.70 (4H, bs), 1.92-0.85 (15H, m); Mass (m/z): 343 (M⁺+1).
3-Azabicyclo[3.2.1]oct-8-yl 2-hydroxy-3-methyl-2-phenylbutanoate (Compound No. 27)
¹H NMR (CDCl₃)δ: 7.69 (2H, d, J=6 Hz), 7.40 (2H, m), 7.29 (1H, m), 4.90 (1H, bs), 3.26 (1H, d), 2.84-2.30 (4H, m), 2.20-1.75 (6H, m), 1.02 (3H, d, J=6 Hz), 0.76 (3H, d, J=6 Hz).
Mass (m/z): 304 (M⁺+1).
3-Azabicyclo[3.2.1]oct-8-ylmethyl hydroxy(diphenyl)acetate (Compound No. 35)
¹H NMR (CDCl₃)δ: 7.43-7.26 (10H, m), 4.05 (2H, t, J=6 Hz), 2.93 (2H, d,J=9 Hz), 2.72 (2H, m), 2.05-1.11 (7H, m).
3-Azabicyclo[3.2.1]oct-8-ylmethyl 2,2-diphenylpropanoate (Compound No. 37)
¹H NMR (CDCl₃)δ: 7.32-7.21 (10H, m), 3.92 (2H, d, J=6 Hz), 2.91 (2H, d, J=9 Hz), 2.75 (2H, d, J=9 Hz), 2.10 (1H, s), 1.99 (3H, s), 1.93 (3H, bs), 1.76 (3H, s); Mass (m/z): 350 (M⁺+1).
3-Azabicyclo[3.2.1]oct-8-ylmethyl methoxy(diphenyl)acetate (Compound No. 45)

### ¹H NMR (CDCl₃)δ: 7.43-7.32 (10H, m), 3.98 (2H, d, J=6 Hz), 3.15 (3H, s), 3.06 (2H, d, J=9 Hz), 2.78 (2H, d, J=9 Hz), 1.96-1.25 (7H, m).

N-(3-Azabicyclo[3.2.1]oct-8-ylmethyl)-2R-[(1S)-3,3-difluorocyclopentyl]-2-hydroxy-2-phenylacetamide (Compound No. 51)
¹H NMR (CDCl₃)δ: 7.57 (2H, d, J=6 Hz), 7.37 (2H, t J=, 6 Hz), 7.31 (1H, d, J=6hz), 6.39 (1H, bs), 3.34 (1H, m), 2.99 (2H, m), 2.80-2.68 (4H, m), 2.16-1.25 (15H, m); Mass (m/z): 379 (M⁺+1)
N-(3-Azabicyclo[3.2.1]oct-8-ylmethyl)-2R-[(1R)-3,3-difluorocyclopentyl]-2-hydroxy-2-phenylacetamide (Compound No. 52)
¹H NMR (CDCl₃)δ: 7.56 (2H, d, J=6 Hz), 7.37 (2H, t, J=6 Hz), 7.31 (1H, d, J=6 Hz), 6.44 (1H, bs), 3.35 (1H, m), 3.03-2.95 (2H, m), 2.65 (4H, m), 2.06-1.26 (15H, m); Mass (m/z): 379 (M⁺+1).
3-Azabicyclo[3.2.1]oct-8-ylmethyl hydroxy[bis(3-methylphenyl)]acetate (Compound No. 56) HPLC: 84.38%

### Example 8

### Synthesis of N-(3-azabicyclo[3.2.1]oct-8-ylmethyl)-2-hydroxy-2-phenyl-2-(2-thienyl)acetamide (Compound No. 28)

To the compound No. 57 (450 mg, 0.9183 mmol) was added methanolic potassium hydroxide solution (30 ml, 40%) and refluxed for 4 hours. The reaction mixture was concentrated under reduced pressure and the residue thus obtained was diluted with water. The aqueous layer was acidified using dilute hydrochloric acid up to pH 3 and impurities were extracted with dichloromethane. The aqueous layer was basified with dilute sodium hydroxide and extracted with ethyl acetate. The organic layer was washed with water and brine, dried over anhydrous sodium sulphate and concentrated under reduced pressure to furnish the title compound. Yield: 195 mg.

¹H NMR (CDCl₃)δ: 7.51 (2H, bs), 7.35 (4H, m), 7.00 (2H, s), 6.52 (1H, bs), 3.49 (1H, s), 3.14 (2H, m), 3.00 (2H, bs), 2.83 (2H, d, J=6 Hz), 2.12-1.13 (7H, m); Mass (m/z): 357.05 (M⁺+1).
Analogues of N-(3-azabicyclo[3.2.1]oct-8-ylmethyl)-2-hydroxy-2-phenyl-2-(2-thienyl)acetamide (Compound No. 28) described below were prepared by deprotecting appropriate compound, respectively, as applicable in each case.
N-(3-azabicyclo[3.2.1]oct-8-ylmethyl)-2-cyclopentyl-2-hydroxy-2-(2-thienyl)acetamide (Compound No. 29)
¹H NMR (CDCl₃)δ: 7.24 (1H, m), 7.09 (1H, bs), 6.97 (1H, bs), 6.47 (1H, bs), 3.55-2.67 (8H, m), 1.98-1.13 (15H, m); Mass (m/z): 349.06 (M⁺+1), 331.05 (M—OH)⁺.

### Example 9

### Synthesis of tartarate salt of 3-azabicyclo[3.2.1]oct-8-ylmethyl hydroxy[bis(3-methylphenyl)]acetate (Compound No. 36)

To a solution of compound no. 56 (60 mg, 0.158 mmol) in ethanol was added L(+) tartaric acid (0.158 mmol) and stirred for 1 hour at 60-70° C. The reaction mixture was concentrated under reduced pressure and the crude product thus obtained was macerated with diethylether and decanted. The residue thus obtained was concentrated under reduced pressure to furnish the title compound. Yield: 64 mg.

¹H NMR (CD₃OD)δ: 7.14-7.03 (8H, m), 4.34 (2H, bs), 3.97 (2H, d, 6 Hz), 3.21 (9H, s), 3.10 (3H, m), 2.22 (6H, s).

### Example 10

### Synthesis of hydrochloride salt of 3-azabicyclo[3.2.1 ]oct-8-yl cyclopentyl (hydroxy)phenylacetate (Compound No. 3)

To a solution of compound No. 2 (10 mg, 0.03 mmol) in dichloromethane (3 ml) and ethanolic hydrochloric acid (5.7 N, 0.2 ml) was added and the reaction mixture was stirred for 5 minutes. The solvent was evaporated under reduced pressure and the residue thus obtained was triturated with hexane to furnish the title compound. Yield=10 mg. m.p: 155° C.

The analogues of hydrochloride salt of 3-azabicyclo[3.2.1]oct-8-yl cyclopentyl(hydroxy) phenylacetate (Compound No. 3) described below, can be prepared by using Compound No. 6 in place of Compound No. 2.
Hydrochloride salt of 3-Azabicyclo[3.2.1]oct-8-yl hydroxy(diphenyl)acetate (Compound No. 11)

### Yield: 80%; m.p: 155.5-155.8° C.

Hydrochloride salt of (2R)-N-[(3-benzyl-3-azabicyclo[3.2.1]oct-8-yl)methyl]-3-hydroxy-2-phenylpropanamide (Compound No. 53)

### ¹H NMR (CDCl₃) δ:7.57-7.24 (m, 10H), 4.16 (bs, 2H), 3.80 (m, 1H), 3.72 (bs, 1H), 3.33 (bs, 1H), 3.01 (bs, 1H), 2.96 (bs, 1H), 2.88 (bs, 1H), 2.47 (bs, 1H), 1.86 (bs, 2H), 1.57-1.21 (m, 6H).

Hydrochloride salt of (2S)-N-[(3-benzyl-3-azabicyclo[3.2.1]oct-8-yl)methyl]-3-hydroxy-2-phenylpropanamide (Compound No. 54)

### ¹H NMR (CD3OD) δ: 7.57-7.24(m, 10H), 5.80(bs, 1H), 4.1(bs, 2H), 3.8-3.7(m, 2H), 3.3(s, 1H), 3.01(s, 1H), 2.47(s, 1H), 2.15(s, 1H), 1.85(s, 2H), 1.57-1.21 (m, 7H).

### Example 11

### Synthesis of 3-[2-(1,3-benzodioxol-5-yl)ethyl]-3-azabicyclo[3.2.1]oct-8-yl cyclopentyl(hydroxy)phenylacetate (Compound No. 4)

To a solution of compound No. 2 (0.3 g, 0.9 mmol) and 5-(2-bromoethyl)-1,3-benzodioxole (0.23 g, 1.0 mmol) in acetonitrile (10.0 ml), was added potassium carbonate (0.38 g, 2.7 mmol) and potassium iodide (0.3 g, 1.8 mmol). The reaction mixture was refluxed for 12 hours. The solvent was concentrated under reduced pressure and the residue thus obtained was diluted with ethyl acetate and water. The ethyl acetate layer was concentrated under reduced pressure. The residue thus obtained was purified by column chromatography using ethyl acetate in hexane as eluent to furnish the title compound. Yield=0.4 g.

IR (DCM): 1721.4 cm⁻¹; ¹H NMR (CDCl₃):δ 7.67-7.70 (m, 2H), 7.23-7.36 (m, 3H), 6.64-6.75 (m, 3H), 5.93 (s, 2H), 4.71-4.74 (m, 1H), 2.98-3.00 (m, 1H), 2.46-2.62 (m, 7H), 2.21 (s, 1H), 2.02-2.04 (m, 2H), 1.39-1.73 (m, 12H); Mass (m/z): 478 (M⁺+1).

The following analogues were prepared similarily,

[3-(4-Methylpent-3-en-1-yl)-3-azabicyclo[3.2.1]oct-8-yl]methyl 2,2-diphenylpropanoate (Compound No. 42)

### ¹H NMR (CDCl₃)δ: 7.33-7.21 (10H, m), 5.01-4.97 (1H, m), 3.96-3.94 (2H, d), 3.65-2.49 (10H, m), 1.94 (3H, s), 1.84-1.68 (6H, m), 0.96-0.82 (4H, m).

3-(4-Methylpent-3-en-1-yl)-3-azabicyclo[3.2.1]oct-8-yl 2-hydroxy-3-methyl-2-phenylbutanoate (Compound No. 40)
¹H NMR (CDCl₃)δ: 7.68-7.67 (2H, m), 7.67-7.30 (3H, m), 5.03 (1H, m), 4.75-4.73 (1H, m), 2.76-2.01 (11H, m), 1.98-1.72 (10H, m), 1.13-1.04 (6H, m); Mass (m/z): 386 (M⁺+1).
2-Cyclopentyl-2-hydroxy-N-{[3-(4-methylpent-3-en-1-yl)-3-azabicyclo[3.2.1]oct-8-yl]methyl}-2-(2-thienyl)acetamide (Compound No. 41)
¹H NMR (CDCl₃)δ: 7.11 (1H, m), 7.10 (1H, m), 6.98-6.97 (1H, m), 6.74 (1H, s), 5.01 (1H, m), 3.31-2.67 (13H, m), 2.18-2.06 (6H, m), 1.89-1.87 (4H, m); Mass (m/z): 431 (M⁺+1).

### Example 12

### Synthesis of 3-methyl-3-azabicyclo[3.2.1]oct-8-yl cyclopentyl(hydroxy) phenylacetate (Compound No. 8)

To a solution of Compound No. 2 (0.4 g, 1.22 mmol) in acetonitrile (20.0 ml) and formaldehyde (37%, 2.6 ml), was added sodium cyanoborohydride (0.26 g, 4.2 mmol) at 25-30° C. The reaction mixture was stirred for 1 hour and subsequently neutralized with acetic acid (1.8 ml). The reaction mixture was again stirred for 12 hours at the same temperature. The solvent was removed under reduced pressure and the residue thus obtained was diluted with water and basified to pH=14 with sodium hydroxide (10%). The reaction mixture was extracted with ethylacetate, dried over anhydrous sodium sulphate and concentrated under reduced pressure. The residue thus obtained was purified by column chromatography using ethyl acetate in hexane as eluent to furnish the title compound. Yield=0.24 g.

IR (DCM): 1722.8 cm⁻¹; ¹H NMR (CDCl₃):δ7.68-7.70 (m, 2H), 7.29-7.38 (m, 3H), 4.70-4.73 (m, 1H), 2.98-3.03 (m, 1H), 2.47-2.51 (m, 2H), 2.19 (m, 1H), 1.97-2.10 (m, 6H), 1.40-1.77 (m, 12H); Mass (m/z): 344 (M⁺+1).

### Example 13

### Synthesis of 3-Benzyl-3-azabicyclo[3.2.1]oct-8-yl 9H-xanthene-9-carboxylate (Compound No. 10)

To a solution of Xanthane-9-carboxylic acid (commercially available) (0.5 g) in dimethylformamide (10 ml), was added carbonyldiimidazole (0.43 g) and the reaction mixture was stirred for 1 hour at 25-30° C. The resulting solution was added to a suspension of 3-benzyl-3-aza-bicyclo[3.2.1]octan-8-ol (0.86 g), sodium hydride (0.11 g) and dimethylformamide (10.0 ml). The reaction mixture was stirred for 12 hours at 25-30° C. and the contents were subsequently poured into water and extracted with ethyl acetate. The organic layer was dried and concentrated under reduced pressure. The residue thus obtained was purified by column chromatography using ethylacetate in hexane as eluent to furnish the title compound. Yield=0.25 g.

### ¹H NMR (CDCl₃):δ7.08-7.35 (m, 13H), 4.97 (s, 1H), 4.55-4.65 (m, 1H), 3.22 (s, 2H), 2.09-2.22 (m, 2H), 1.93 (m, 2H), 1.80-1.85 (m, 2H), 1.54-1.65 (m, 4H); Mass(m/z): 426 (M⁺+1).

### Example 14

### Synthesis of 3-azabicyclo[3.2.1]oct-8-yl 9H-xanthene-9-carboxylate (Compound No. 9)

The title compound was prepared following the procedure as described for the synthesis of compound No. 2, Example 2 by using 9H-Xanthene-9-carboxylic acid-3-benzyl-3-aza-bicyclo[3.2.1]oct-8-ylester in place of compound No. 1. Yield=65%.

IR (DCM): 1738.7 cm⁻¹; ¹H NMR (CDCl₃):δ7.14-7.40 (m, 8H), 5.02 (s, 1H), 4.68-4.71 (m, 1H), 2.74-2.89 (m, 4H), 2.05-2.20 (m, 4H), 1.82-1.85 (m, 2H); Mass (m/z): 336 (M⁺+1).

### Biological Activity

### Radioligand Binding Assays:

The affinity of test compounds for M₂ and M₃ muscarinic receptor subtypes was determined by [³H]-N-Methylscopolamine (NMS) binding studies using rat heart and submandibular gland respectively as described by Moriya et al., (Life Sci., 1999, 64(25): 2351-2358) with minor modifications. Specific binding of [³H]-NMS was also determined using membranes from Chinese hamster ovary (CHO) cells expressing cloned human muscarinic receptor subtypes.

### Membrane Preparation:

(a) Rat tissues Submandibular glands and heart were isolated and placed in ice-cold homogenising buffer (HEPES 20 mM, 10 mM EDTA, pH 7.4) immediately after sacrifice. The tissues were homogenised in ten volumes of homogenising buffer and the homogenate was filtered through two layers of wet gauze and filtrate was centrifuged at 500 g for 10 min at 4° C. The supernatant was subsequently centrifuged at 40,000 g for 20 min at 4° C. The pellet thus obtained was resuspended in assay buffer (HEPES 20 mM, EDTA 5 mM, pH 7.4) and were stored at −70° C. until the time of assay.
(b) CHO cells expressing human recombinant receptors The cell pellets were homogenised for 30 sec at 12,000 to 14,000 rpm, with intermittent gaps of 10-15 sec in ice-cold homogenising buffer (20 mM HEPES, 10 mM EDTA, pH 7.4). The homogenate was then centrifuged at 40,000 g for 20 min at 4° C. The pellet thus obtained was re suspended in homogenising buffer containing 10% sucrose and was stored at −70° C. until the time of assay.

### Ligand Binding Assay:

The compounds were dissolved and diluted in dimethyl sulphoxide. The membrane homgenates (5-10 μg protein) were incubated in 250 μL of assay buffer (20 mM HEPES, pH 7.4) at 24-25° C. for 3 hrs. Non-specific binding was determined in the presence of 1 μM Atropine. The incubation was terminated by vacuum filtration over GF/B fiber filter mats (Wallac) using Skatron cell harvester. The filters were then washed with ice-cold 50 mM Tris HCl buffer (pH 7.4). The filter mats were dried and transferred to 24 well plates (PET A No Cross Talk) followed by addition of 500 μl of scintillation cocktail. Radioactivity retained on filters was counted in Microbeta scintillation counter. The IC₅₀ & Kd were estimated by using the non-linear curve-fitting program using GraphPad Prism software. The value of inhibition constant, Ki was calculated from competitive binding studies by using Cheng & Prusoff's equation (Biochem Pharmacol, 1973,22: 3099-3108), Ki=IC₅₀/(1+[L]/Kd), where [L] is the concentration of ligand [³H]-N-methyl scopolamine used in the particular experiment and Kd is the estimate of affinity of receptors to the ligand.

Above specifically disclosed compounds (Nos. 1-48) exhibited Kᵢ values for rat M₂ muscarinic receptors in the range of about 0.6 nM to over 1000 nM, for example from about 0.6 nM to about 150 nM, or for example from about 0.6 nM to about 25 nM, or for example from about 0.6 nM to about 8 nM (as compared to about 5 nM for tolteridine). Above specifically disclosed compounds (Nos. 1-48) exhibited Kᵢ values for rat M₃ muscarinic receptors in the range of about 0.8 nM to over 1000 nM, for example from about 0.8 nM to about 150 nM, or for example from about 0.8 nM to about 25 nM, or for example from about 0.8 nM to about 7 nM (as compared to about 4 nM for tolteridine).

Particular specifically disclosed compounds (Nos. 49-54) exhibited Kᵢ values for human M₂ muscarinic receptors in the range of about 9 nM to about 130 nM, for example from about 9 nM to about 35 nM, or for example from about 9 nM to about 20 nM (as compared to about 5 nM for tolteridine). Above specifically disclosed compounds (Nos. 49-54) exhibited Kᵢ values for human M₃ muscarinic receptors in the range of about 70 nM to about 700 nM, for example from about 70 nM to about 500 nM, or for example from about 70 nM to about 220 nM, or for example from about 70 nM to about 155 nM (as compared to about 6 nM for tolteridine).

While the present invention has been described in terms of its specific embodiments, certain modification and equivalents will be apparent to those skilled in the art and are intended to be included within the scope of the present invention.

We claim:

1. A compound of Formula I, 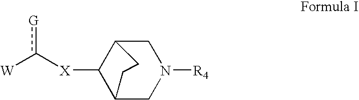
and its enantiomers, diastereomers, N-oxides, polymorphs, pharmaceutically acceptable salts and pharmaceutically acceptable solvates, wherein: [symbol] represents an optional double bond;
W is 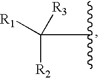
heteroaryl, or heterocyclyl, where [symbol] represents a point of attachment;
R₁ is aryl, heteroaryl, heterocyclyl, or cycloalkyl;
R₂ is alkyl, alkenyl, alkynyl, cycloalkyl, aryl, heteroaryl, heterocyclyl, heteroarylalkyl, or heterocyclylalkyl;
R₃ is hydrogen, lower alkyl, hydroxy, alkoxy, alkenyloxy, alkynyloxy, halogen, or amino;
X is oxygen, sulphur, or alkylene, wherein the alkylene may be interrupted by 1-5 oxygen, sulfur and —NRₐ (where Rₐ can be hydrogen, alkyl, cycloalkyl, alkenyl, alkynyl, or aryl) groups;
R₄ is hydrogen; alkyl; alkenyl; alkynyl; cycloalkyl; carboxy; halogen; aryl ; aralkyl; acyl; heteroaryl; heterocyclyl; SO₂R₅ [wherein R₅ is selected from alkyl, alkenyl, alkynyl, cycloalkyl, —NRₚR_{q} (wherein Rₚ and R_{q} are selected from hydrogen, alkyl, alkenyl, alkynyl, cycloalkyl, aryl, aralkyl, heterocyclyl, heteroaryl, heterocyclylalkyl, or heteroarylalkyl, or Rₚ and R_{q} may also together join to form a heterocyclyl ring), aryl, aralkyl, heteroaryl, heterocyclyl, heterocyclylalkyl, or heteroarylalkyl]; —COOR₆ (wherein R₆ is selected from alkyl, alkenyl, alkynyl, cycloalkyl, aryl, or aralkyl); —(C═O)NRₓR_{y} [wherein Rₓ and R_{y} are selected from hydrogen, hydroxy (as restricted by the definition that both Rₓ and R_{y} cannot be hydroxy at the same time), alkyl, alkenyl, alkynyl, aryl, aralkyl, SO₂R₅ wherein R₅ is the same as defined above, heteroaryl, heterocyclyl, heteroarylalkyl, or heterocyclylalkyl, or Rₓ and R_{y} may also together join to form a heterocyclyl ring]; —NRₓR_{y} wherein Rₓ and R_{y} are the same as defined above; and —O—C(═O)NRₓR_{y} wherein Rₓ and R_{y} are the same as defined above; and
G is —OR (wherein R represents hydrogen or unsubstituted lower (C₁-C₆) alkyl); —NOR (wherein R is the same as defined above); —NHYR′ (wherein R′ is hydrogen, alkyl or aryl and
Y is —C(═O), SO, SO₂); or oxygen,
with the proviso that when X is oxygen or sulphur, then G can not be OR (wherein R is defined above).

2. The compound of claim 1, selected from
3-Benzyl-3-azabicyclo[3.2.1]oct-8-yl cyclopentyl(hydroxy)phenylacetate (Compound No. 1),
3-Azabicyclo[3.2.1]oct-8-yl cyclopentyl(hydroxy)phenylacetate (Compound No. 2),
3-Azabicyclo[3.2.1]oct-8-ylcyclopentyl(hydroxy)phenylacetate (Compound No. 3),
3-[2-(1,3-Benzodioxol-5-yl)ethyl]-3-azabicyclo[3.2.1]oct-8-yl cyclopentyl(hydroxy) phenylacetate (Compound No. 4),
3-Benzyl-3-azabicyclo[3.2.1]oct-8-yl hydroxy(diphenyl)acetate (Compound No. 5),
3-Azabicyclo[3.2.1]oct-8-yl hydroxy(diphenyl)acetate (Compound No. 6),
3-Benzyl-3-azabicyclo[3.2.1]oct-8-yl cyclohexyl(hydroxy)phenylacetate (Compound No. 7),
3-Methyl-3-azabicyclo[3.2.1]oct-8-yl cyclopentyl(hydroxy)phenylacetate (Compound No. 8),
3-Azabicyclo[3.2.1]oct-8-yl 9H-xanthene-9-carboxylate (Compound No. 9),
3-Benzyl-3-azabicyclo[3.2.1]oct-8-yl 9H-xanthene-9-carboxylate (Compound No. 10),
Hydrochloride salt of 3-Azabicyclo[3.2.1]oct-8-yl hydroxy(diphenyl)acetate (Compound No. 11),
3-Benzyl-3-azabicyclo[3.2.1]oct-8-yl cyclopentyl(hydroxy)(4-methylphenyl)acetate (Compound No. 12),
3-Benzyl-3-azabicyclo[3.2.1]oct-8-yl cyclopentyl(hydroxy)2-thienylacetate (Compound No. 13),
3-Azabicyclo[3.2.1]oct-8-yl cyclopentyl(hydroxy)(4-methylphenyl)acetate (Compound No. 14),
N-(3-Azabicyclo[3.2.1]oct-8-ylmethyl)-2-hydroxy-2,2-diphenylacetamide (Compound No. 15),
N-3-benzyl-3-azabicyclo [3.2.1]oct-8-yl]methyl}-2-hydroxy-2,2-diphenylacetamide (Compound No. 16),
3-Benzyl-3-azabicyclo[3.2.1]oct-8-yl hydroxy(phenyl)2-thienylacetate (Compound No. 17),
3-Benzyl-3-azabicyclo[3.2.1]oct-8-yl cyclopentyl(4-fluorophenyl)hydroxyacetate (Compound No. 18),
N-[(3-benzyl-3-azabicyclo[3.2.1]oct-8-yl)methyl]-2-cyclopentyl-2-hydroxy-2-phenylacetamide (Compound No. 19),
N-[(3-benzyl-3-azabicyclo[3.2.1]oct-8-yl)methyl]-2-cyclopentyl-2-hydroxy-2-(2-thienyl)acetamide (Compound No. 20),
N-[(3-benzyl-3-azabicyclo[3.2.1]oct-8-yl)methyl]-2-hydroxy-2-phenyl-2-(2-thienyl)acetamide (Compound No. 21),
3-Benzyl-3-azabicyclo[3.2.1]oct-8-yl 2-hydroxy-3-methyl-2-phenylbutanoate (Compound No. 22),
N-[(3-benzyl-3-azabicyclo[3.2.1]oct-8-yl)methyl]-3-hydroxy-2-phenylpropanamide (Compound No. 23),
(3-Benzyl-3-azabicyclo[3.2.1]oct-8-yl)methyl cyclopentyl(hydroxy)phenylacetate (Compound No. 24),
3-Benzyl-3-azabicyclo[3.2.1]oct-8-yl 2-(4-fluorophenyl)-2-hydroxy-3-methylbutanoate Compound No. 25),
N-(3-Azabicyclo[3.2.1]oct-8-ylmethyl)-2-cyclopentyl-2-hydroxy-2-phenylacetamide (Compound No. 26),
3-Azabicyclo[3.2.1]oct-8-yl 2-hydroxy-3-methyl-2-phenylbutanoate (Compound No. 27),
N-(3-azabicyclo[3.2.1]oct-8-ylmethyl)-2-hydroxy-2-phenyl-2-(2-thienyl)acetamide (Compound No. 28),
N-(3-azabicyclo[3.2.1]oct-8-ylmethyl)-2-cyclopentyl-2-hydroxy-2-(2-thienyl)acetamide (Compound No. 29),
(3-Benzyl-3-azabicyclo[3.2.1]oct-8-yl)methyl cyclopentyl(hydroxy)2-thienylacetate (Compound No. 30),
(3-Benzyl-3-azabicyclo[3.2.1]oct-8-yl)methyl hydroxy(phenyl)2-thienylacetate (Compound No. 31),
(3-Benzyl-3-azabicyclo[3.2.1]oct-8-yl)methyl hydroxy(diphenyl)acetate (Compound No. 32),
(3-Benzyl-3-azabicyclo[3.2.1]oct-8-yl)methyl hydroxy[bis(3-methylphenyl)]acetate (Compound No. 33),
(3-Benzyl-3-azabicyclo[3.2.1]oct-8-yl)methyl 2,2-diphenylpropanoate (Compound No. 34),
3-Azabicyclo[3.2.1]oct-8-ylmethyl hydroxy(diphenyl)acetate (Compound No. 35),
Tartarate salt of 3-azabicyclo[3.2.1]oct-8-ylmethyl hydroxy[bis(3-methylphenyl)]acetate (Compound No. 36),
3-Azabicyclo[3.2.1]oct-8-ylmethyl 2,2-diphenylpropanoate (Compound No. 37), (3-benzyl-3-azabicyclo[3.2.1]oct-8-yl)methyl methoxy(diphenyl)acetate (Compound No. 38),
N-[(3-benzyl-3-azabicyclo[3.2.1]oct-8-yl)methyl]-2-methoxy-2,2-diphenylacetamide (Compound No. 39),
3-(4-Methylpent-3-en-1-yl)-3-azabicyclo[3.2.1]oct-8-yl 2-hydroxy-3-methyl-2-phenylbutanoate (Compound No. 40),
2-Cyclopentyl-2-hydroxy-N- {[3-(4-methylpent-3-en-1-yl)-3-azabicyclo[3.2.1]oct-8-yl]methyl}-2-(2-thienyl)acetamide (Compound No. 41),
[3-(4-Methylpent-3-en-1-yl)-3-azabicyclo[3.2.1]oct-8-yl]methyl 2,2-diphenylpropanoate (Compound No. 42),
(4E, 4Z)-N-[(3-benzyl-3-azabicyclo[3.2.1]oct-8-yl)methyl]-2-hydroxy-2-phenylhex-4-enamide (Compound No. 43),
N-[(3-Benzyl-3-azabicyclo[3.2.1]oct-8-yl)methyl]-2-hydroxy-2,2-bis(3-methylphenyl) acetamide (Compound No. 44),
3-Azabicyclo[3.2.1]oct-8-ylmethyl methoxy(diphenyl)acetate (Compound No. 45),
2-(Allyloxy)-N-[(3-benzyl-3-azabicyclo[3.2.1]oct-8-yl)methyl]-2,2-diphenylacetamide (Compound No. 46),
N-[(3-Benzyl-3-azabicyclo[3.2.1]oct-8-yl)methyl]-2,2-diphenyl-2-propoxyacetamide (Compound No. 47),
(3-Benzyl-3-azabicyclo[3.2.1]oct-8-yl)methyl hydroxy(phenyl)pyridin-3-ylacetate (Compound No. 48),
N-[(3-Benzyl-3-azabicyclo[3.2.1]oct-8-yl)methyl]-2R-((1R)-3,3-difluorocyclopentyl)-2-hydroxy-2-phenylacetamide (Compound No. 49),
N-[(3-Benzyl-3-azabicyclo[3.2.1]oct-8-yl)methyl]-2R-[(1S)-3,3-difluorocyclopentyl]-2-hydroxy-2-phenylacetamide (Compound No. 50),
N-(3-Azabicyclo [3.2.1]oct-8-ylmethyl)-2R-[(1S)-3,3-difluorocyclopentyl]-2-hydroxy-2-phenylacetamide (Compound No. 51),
N-(3-Azabicyclo[3.2.1]oct-8-ylmethyl)-2R-[(1R)-3,3-difluorocyclopentyl]-2-hydroxy-2-phenylacetamide (Compound No. 52),
Hydrochloride salt of (2R)-N-[(3-benzyl-3-azabicyclo[3.2.1]oct-8-yl)methyl]-3-hydroxy-2-phenylpropanamide (Compound No. 53),
Hydrochloride salt of (2S)-N-[(3-benzyl-3-azabicyclo[3.2.1]oct-8-yl)methyl]-3-hydroxy-2-phenylpropanamide (Compound No. 54),
(2S)-N-[(3-benzyl-3-azabicyclo[3.2.1]oct-8-yl)methyl]-3-hydroxy-2-phenylpropanamide (Compound No. 55),
3-Azabicyclo[3.2.1]oct-8-ylmethyl hydroxy[bis(3-methylphenyl)]acetate (Compound No. 56),
Benzyl 8-({[hydroxy(phenyl)2-thienylacetyl]amino}methyl)-3-azabicyclo[3.2.1]octane-3-carboxylate (Compound No. 57),
Benzyl 8-{[cyclopentyl(hydroxy)2-thienylacetyl]oxy}-3-azabicyclo[3.2.1]octane-3-carboxylate (Compound No. 58),
(2R)-N-[(3-benzyl-3-azabicyclo[3.2.1]oct-8-yl)methyl]-3-hydroxy-2- phenylpropanamide(Compound No. 59), and
physiologically acceptable salts thereof.

3. A pharmaceutical composition for the treatment of diseases of the respiratory, urinary and gastrointestinal systems comprising a compound of claim 1, together with a pharmaceutically acceptable carrier.

4. A pharmaceutical composition for the treatment of diseases of the respiratory, urinary and gastrointestinal systems comprising a compound of claim 2, together with a pharmaceutically acceptable carrier.

5. A method for treatment or prophylaxis of an animal or human suffering from a disease or disorder of the respiratory, urinary or gastrointestinal system, wherein the disease or disorder is mediated through muscarinic receptors, the method comprising administration of a therapeutically effective amount of at least one compound of claim 1.

6. A method for treatment or prophylaxis of an animal or human suffering from a disease or disorder associated with muscarinic receptors, the method comprising administration of a therapeutically effective amount of at least one compound of claim 1.

7. The method of claim 6, wherein the disease or disorder is selected from bronchial asthma, chronic obstructive pulmonary disorders (COPD), pulmonary fibrosis, urinary incontinence, lower urinary tract symptoms (LUTS), irritable bowel syndrome, obesity, diabetes and gastrointestinal hyperkinesis.

8. A method for making a compound of Formula VI, 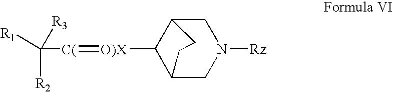
wherein
R₁ is aryl, heteroaryl, heterocyclyl, or cycloalkyl;
R₂ is alkyl, alkenyl, alkynyl, cycloalkyl, aryl, heteroaryl, heterocyclyl, heteroarylalkyl, or heterocyclylalkyl;
R₃ is hydrogen, lower alkyl, hydroxy, alkoxy, alkenyloxy, alkynyloxy, halogen, or amino; and
R_{z} is alkyl or acyl,
the method comprising:
a) reacting a compound of Formula II (wherein R₁, R₂ and R₃ are as defined, Rₙ is alkyl, and P is a protecting group) with a compound of Formula III to give a compound of Formula IV; 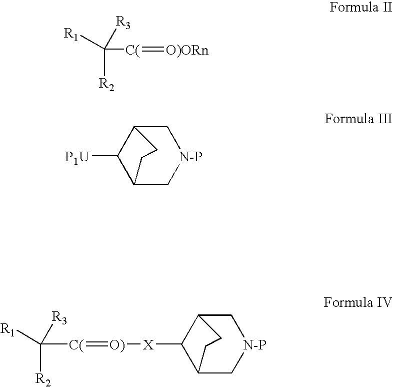
b) deprotecting the compound of Formula IV to give a compound of Formula V; and 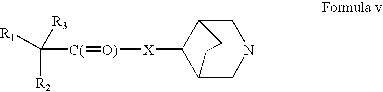
c) reacting the compound of Formula V with a compound of Formula R_{z}-hal (wherein R_{z} is as defined, and hal is halogen) to give the compound of Formula VI.

9. A method for making a compound of Formula VII 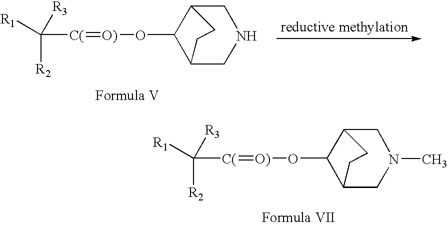
wherein
R₁ is aryl, heteroaryl, heterocyclyl, or cycloalkyl;
R₂ is alkyl, alkenyl, alkynyl, cycloalkyl, aryl, heteroaryl, heterocyclyl, heteroarylalkyl, or heterocyclylalkyl; and
R₃ is hydrogen, lower alkyl, hydroxy, alkoxy, alkenyloxy, alkynyloxy, halogen, or amino; the method comprising: 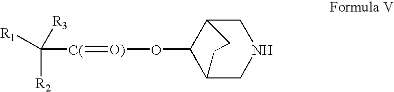
reductively methylating a compound of Formula V to give a compound of Formula VI.

10. A method of making a compound of Formula Va 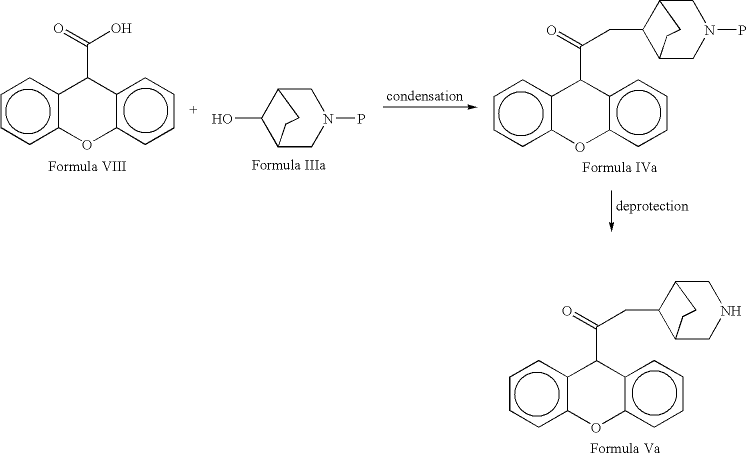
the method comprising:
a) reacting a compound of Formula VIII with a compound of Formula IIIa (wherein P is a protecting group) to give a compound of Formula IVa; and
b) deprotecting the compound of Formula IVa to give the compound of Formula Va.

11. A compound of claim 1, wherein [symbol] represents a double bond;
G is oxygen; and X is oxygen.
